(19) United States
(12) Patent Application Publication
(10) Pub. No.: US 2025/0195203 A1
(43) Pub. Date: Jun. 19, 2025

(54) COMPRESSIBLE-GAS-FILLED IMPLANT WITH LIQUID BUFFER

(71) Applicant: EDWARDS LIFESCIENCES CORPORATION, Irvine, CA (US)

(72) Inventors: Amir Davidesko, Binyamina Givat Ada (IL); Roy Shitrit, Zichron Yaakov (IL)

(73) Assignee: EDWARDS LIFESCIENCES CORPORATION, Irvine, CA (US)

(21) Appl. No.: 19/071,912

(22) Filed: Mar. 6, 2025

Related U.S. Application Data
(63) Continuation of application No. PCT/US2023/075876, filed on Oct. 3, 2023.
(60) Provisional application No. 63/378,467, filed on Oct. 5, 2022.

(51) Int. Cl. A61F 2/06

(52) U.S. Cl. CPC A61F 2/06; A61F 2002/068; A61F 2230/0065

(57) ABSTRACT

A compliance-enhancing implant device dimensioned for deployment within a blood vessel includes a first sealed chamber containing a compressible gas medium and one or more second sealed chambers separated from the first sealed chamber by one or more first membrane portions. The one or more second sealed chambers contain an at least partially liquid medium. The first sealed chamber is surrounded on all sides by the one or more second sealed chambers. A portion of the compressible gas medium in the first sealed chamber can pass through the first membrane portion into the at least partially liquid medium in the one or more second sealed chambers.

### RELATED APPLICATION(S)

This application is a continuation of International Patent Application No. PCT/PCT/US23/75876, filed Oct. 3, 2023, which claims the benefit of U.S. Provisional patent application Ser. No. 63/378,467, filed on Oct. 5, 2022, the complete disclosures of which are hereby incorporated by reference in its entirety.

### BACKGROUND

The present disclosure generally relates to the field of medical implant devices. Insufficient or reduced compliance in certain blood vessels, including arteries such as the aorta, can result in reduced perfusion, cardiac output, and other health complications. Restoring compliance and/or otherwise controlling flow in such blood vessels can improve patient outcomes.

### SUMMARY

Described herein are devices, methods, and systems that facilitate the restoration of compliance characteristics to undesirably stiff blood vessels. Devices associated with the various examples of the present disclosure can include an implant device comprising a first chamber containing compressible gas and a second chamber containing liquid, the second chamber being separated from the first chamber by a membrane. When the implant device is implanted in a blood vessel, the compressible gas chamber can compress and expand in response to changing pressure conditions to thereby provide a change in volume over the cardiac cycle. Such change in volume can level-out the pressure and/or flow waveforms of the blood vessel and/or promote blood flow during, for example, the diastolic phase of the cardiac cycle.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 illustrates an example representation of cardiac and vascular anatomy of a patient.

FIG. 2A shows an example healthy aorta.

FIGS. 2B and 2C show side and axial cross-sectional views, respectively, of the healthy aorta of FIG. 2A experiencing compliant expansion and contraction over a cardiac cycle,

FIG. 3A shows an example stiff aorta.

FIGS. 3B and 3C show side and axial cross-sectional views, respectively, of the stiff aorta of FIG. 3A experiencing compromised expansion and contraction over a cardiac cycle.

FIGS. 4-1 and 4-2 show an axial compressible compliance-enhancing implant device in expanded and compressed configurations, respectively, in accordance with one or more examples.

FIGS. 5-1 and 5-2 show a tubular compressible compliance-enhancing implant device in expanded and compressed configurations, respectively, in accordance with one or more examples.

FIG. 6-1 shows a gas-filled chamber and a liquid-filled chamber separated by a membrane in accordance with one or more examples.

FIG. 6-2 shows the gas-filled chamber and liquid-filled chamber of FIG. 6-1 after some amount of diffusion of molecules has occurred from the gas-filled chamber into the liquid-filled chamber through the membrane in accordance with one or more examples.

FIGS. 7A-7D provide views of a compliance-enhancing implant device including one or more compressible gas-filled chambers buffered from the exterior of the device by one or more liquid-filled chambers in accordance with one or more examples.

FIGS. 8-1 and 8-2 show side cross-sectional views of the compliance-enhancing implant device of FIGS. 7A-7D in compressed and relaxed configurations, respectively, in accordance with one or more examples.

FIG. 9 shows a side cross-sectional view of a compliance-enhancing device having non-elastic sidewalls in accordance with one or more examples.

FIGS. 10-1, 10-2, 10-3, and 10-4 illustrate a flow diagram for a process for implanting a compliance-enhancing implant device in accordance with one or more examples.

FIGS. 11-1, 11-2, 11-3, and 11-4 provide images of the compliance implant device and certain anatomy corresponding to operations of the process of FIGS. 10-1, 10-2, 10-3, and 10-4 according to one or more examples.

FIG. 12 shows a compliance-enhancing implant device deployed in a pulmonary artery in accordance with one or more examples.

FIGS. 13A, 13B, and 13C provide side, side cross-sectional, and axial views, respectively, of an axial compliance-enhancing implant device including a compressible-gas-filled chamber and a liquid-filled buffer chamber in accordance with one or more example.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to only the numeric portion (e.g., ‘10’) may refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present written description to a feature ‘10’ may be understood to refer to either an identified feature ‘10a’ in a particular figure of the present disclosure or to an identifier ‘10’ or ‘10b’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

Examples of the present disclosure relate to inventive implant devices configured to enhance the compliance and/or other flow characteristics of a blood vessel, such as the aorta or portion thereof. Such devices, in some instances, comprise compressible-gas-filled sleeves and/or chambers, wherein such gas-filled compartments are buffered from systemic circulation by one or more liquid-filled compartments, which can prevent, and/or reduce the risks associated with, gas depletion from the gas-filled compartment(s) via diffusion into the bloodstream. Example implant devices of the present disclosure can include double-walled balloon devices, wherein a wall or membrane separates gas-filled and liquid-filled compartments. Ambient pressures greater than certain threshold(s) apply forces on the device to compress the gas contained therein, such that the implant device changes volume as environmental pressure conditions change. Example implant devices of the present disclosure can be implemented to reduce pulsatile left ventricular afterload, which may be helpful for treating patients experiencing hypertensive heart failure with preserved ejection fraction, and or other conditions.

### Vascular Anatomy and Compliance

Certain examples are disclosed herein in the context of vascular implant devices, and in particular, compliance-enhancement implant devices implanted in the aorta. However, although certain principles disclosed herein may be particularly applicable to the anatomy of the aorta, it should be understood that compliance-enhancement implant devices in accordance with the present disclosure may be implanted in, or configured for implantation in, any suitable or desirable blood vessels or other anatomy, such as the inferior vena cava.

The anatomy of the heart and vascular system is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., ventricles, pulmonary artery, aorta, etc.). The contraction of the various heart muscles may be prompted by signals generated by the electrical system of the heart.

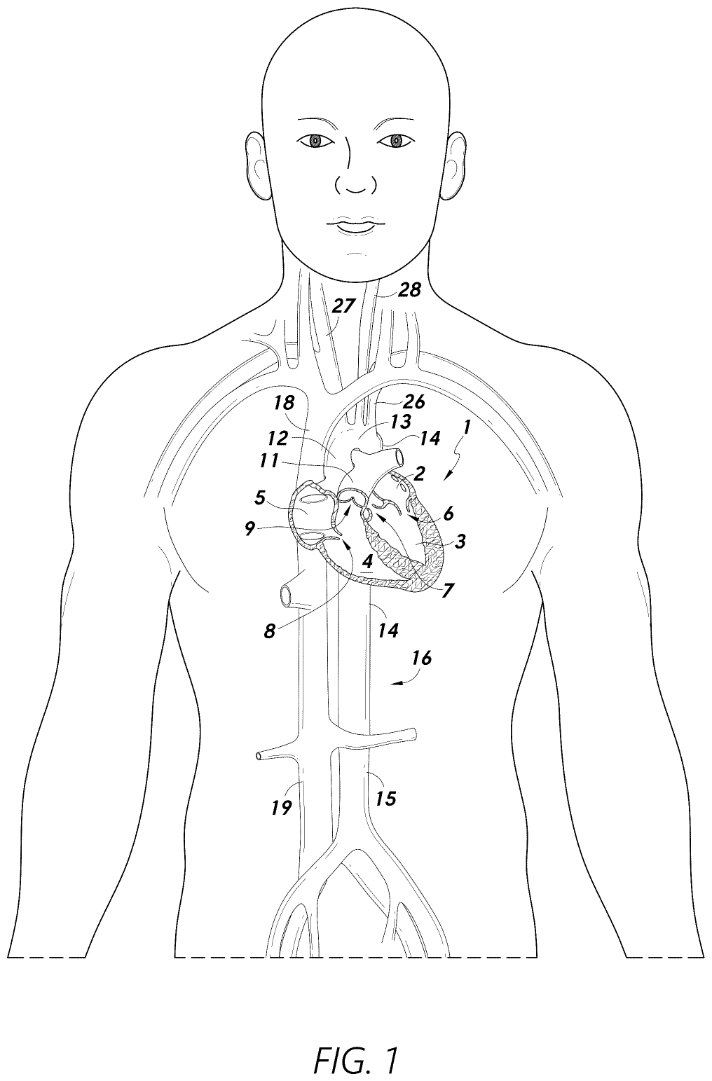

FIG. 1 illustrates an example representation of a heart 1 and associated vasculature having various features relevant to one or more examples of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery 11 via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs. The pulmonary artery 11 includes a pulmonary trunk and left and right pulmonary arteries that branch off of the pulmonary trunk, as shown.

The tricuspid valve 8 separates the right atrium 5 from the right ventricle 4. The tricuspid valve 8 generally has three cusps/leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Disfunction of a heart valve and/or associated leaflets (e.g., pulmonary valve disfunction) can result in valve leakage and/or other health complications.

The vasculature of the human body, which may be referred to as the circulatory system, cardiovascular system, or vascular system, contains a complex network of blood vessels with various structures and functions and includes various veins (venous system) and arteries (arterial system). Generally, arteries, such as the aorta 16, carry blood away from the heart, whereas veins, such as the inferior 19 and superior 18 venae cavae, carry blood back to the heart.

The aorta 16 is a compliant arterial blood vessel that buffers and conducts pulsatile left ventricular output and contributes the largest component of total compliance of the arterial tree. The aorta 16 includes the ascending aorta 12, which begins at the opening of the aortic valve 7 in the left ventricle of the heart. The ascending aorta 12 and pulmonary trunk 11 twist around each other, causing the aorta 12 to start out posterior to the pulmonary trunk 11, but end by twisting to its right and anterior side. Among the various segments of the aorta 16, the ascending aorta 12 is relatively more frequently affected by aneurysms and dissections, often requiring open heart surgery to be repaired. The transition from ascending aorta 12 to aortic arch 13 is at the pericardial reflection on the aorta 16. At the root of the ascending aorta 12, the lumen has three small pockets between the cusps of the aortic valve and the wall of the aorta, which are called the aortic sinuses or the sinuses of Valsalva. The left aortic sinus contains the origin of the left coronary artery and the right aortic sinus likewise gives rise to the right coronary artery. Together, these two arteries supply the heart.

As mentioned above, the aorta 16 is coupled to the heart 1 via the aortic valve 7, which leads into the ascending aorta 12 and gives rise to the innominate artery 27, the left common carotid artery 28, and the left subclavian artery 26 along the aortic arch 13 before continuing as the descending thoracic aorta 14 and further the abdominal aorta 15. References herein to the aorta may be understood to refer to the ascending aorta 12 (also referred to as the “ascending thoracic aorta”), aortic arch 13, descending or thoracic aorta 14 (also referred to as the “descending thoracic aorta”), abdominal aorta 15, or other arterial blood vessel or portion thereof.

Arteries, such as the aorta 16, may utilize blood vessel compliance (e.g., arterial compliance) to store and release energy through the stretching of blood vessel walls. The term “compliance” is used herein according to its broad and ordinary meaning, and may refer to the ability of an arterial blood vessel or prosthetic implant device to distend, expand, stretch, or otherwise deform in a manner as to increase in volume in response to increasing intraluminal pressure, and/or the tendency of a blood vessel (e.g., artery) or prosthetic implant device, or portion thereof, to recoil toward its original dimensions as intraluminal pressure decreases.

Arterial compliance facilitates perfusion of organs in the body with oxygenated blood from the heart. Generally, a healthy aorta and other major arteries in the body are at least partially elastic and compliant, such that they can act as a reservoir for blood, filling up with blood when the heart contracts during systole and continuing to generate pressure and push blood to the organs of the body during diastole.

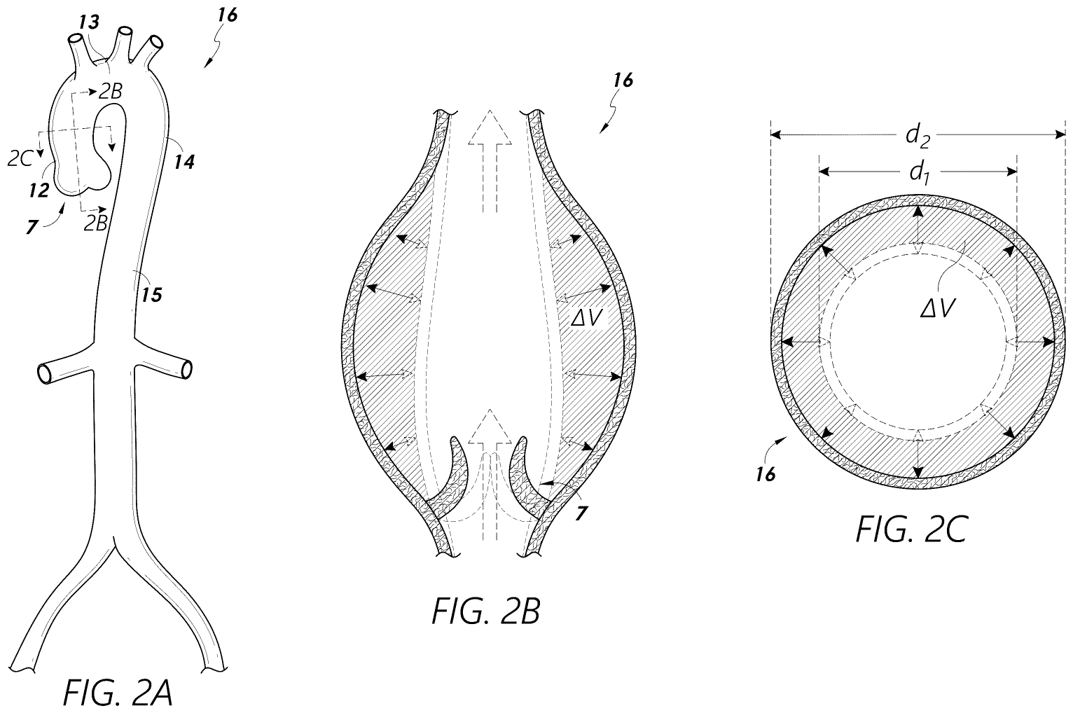

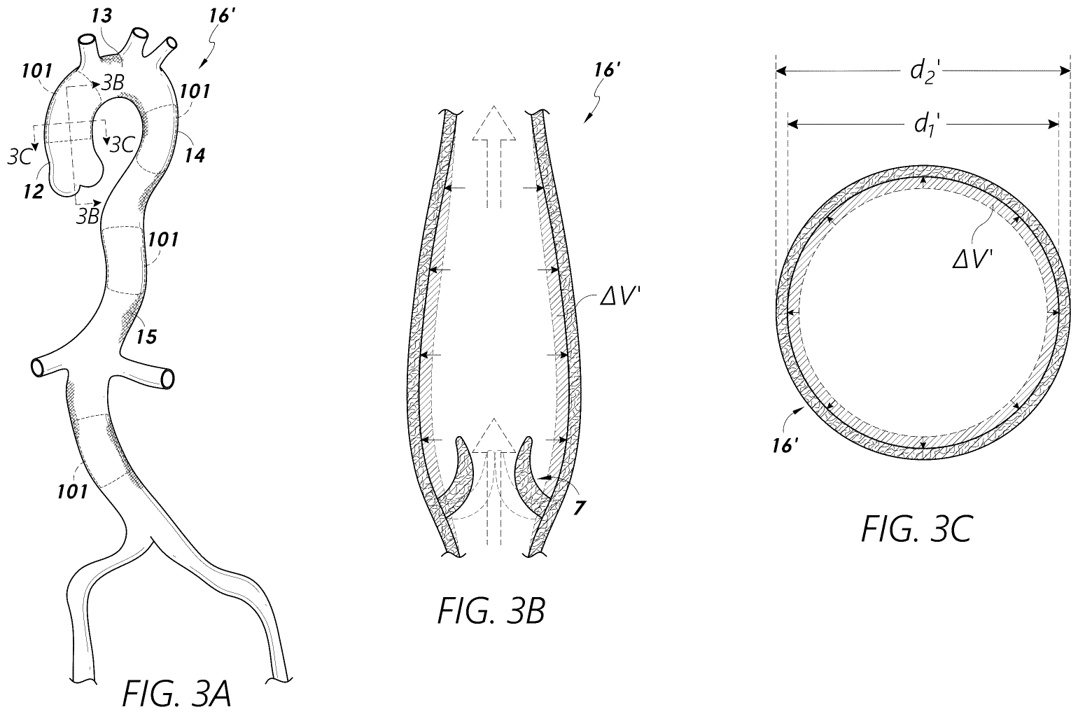

FIG. 2A shows an example healthy aorta 16. FIGS. 2B and 2C show side and axial cross-sectional views, respectively, of the healthy aorta 16 of FIG. 2A experiencing compliant expansion and contraction over a cardiac cycle.

As referenced above, the systolic phase of the cardiac cycle is associated with the pumping phase of the left ventricle, while the diastolic phase of the cardiac cycle is associated with the resting or filling phase of the left ventricle. As shown in FIGS. 2A and 2B, with proper arterial compliance, an increase in volume Δv will generally occur in an artery when the pressure in the artery is increased from diastole to systole. As blood is pumped into the aorta 16 through the aortic valve 7, the pressure in the aorta increases and the diameter of at least a portion thereof expands. A first portion of the blood entering the aorta 16 during systole may pass through the artery during the systolic phase, while a second portion (e.g., approximately half of the total blood volume) may be stored in the expanded volume Δv caused by compliant stretching of the blood vessel 16 from a non-expanded diameter d₁ to an expanded diameter d₂, thereby storing energy for contributing to perfusion during the diastolic phase. A compliant aorta may generally stretch with each heartbeat, such that the diameter of at least a portion of the aorta expands.

The tendency of the arteries to stretch in response to pressure as a result of arterial compliance may have a significant effect on perfusion and/or blood pressure in some patients. For example, arteries with relatively higher compliance may be conditioned to more easily deform than lower-compliance arteries under the same pressure conditions. Compliance (C) may be calculated using the following equation, where Δv is the change in volume (e.g., in mL) of the blood vessel, and Δp is the pulse pressure from systole to diastole (e.g., in mmHg):

$$C = \frac{Δ v}{Δ p} \qquad ( 1 )$$

In older individuals and patients suffering from heart failure and/or atherosclerosis, compliance of the aorta and other arteries can be diminished to some degree or lost. Such reduction in compliance can reduce the supply of blood to the organs of the body due to the decrease in blood flow during diastole. Among the risks associated with insufficient arterial compliance, a significant risk presented in such patients is a reduction in blood supply to the heart muscle itself. For example, during systole, generally little or no blood may flow in the coronary arteries and into the heart muscle due to the contraction of the heart which holds the heart at relatively high pressures. During diastole, the heart muscle generally relaxes and allows flow into the coronary arteries. Therefore, perfusion of the heart muscle relies on diastolic flow, and therefore on aortic/arterial compliance.

Insufficient perfusion of the heart muscle can lead to and/or be associated with heart failure. Heart failure is a clinical syndrome characterized by certain symptoms, including breathlessness, ankle swelling, fatigue, and others. Heart failure may be accompanied by certain signs, including elevated jugular venous pressure, pulmonary crackles and peripheral edema, for example, which may be caused by structural and/or functional cardiac abnormality. Such conditions can result in reduced cardiac output and/or elevated intra-cardiac pressures at rest or during stress.

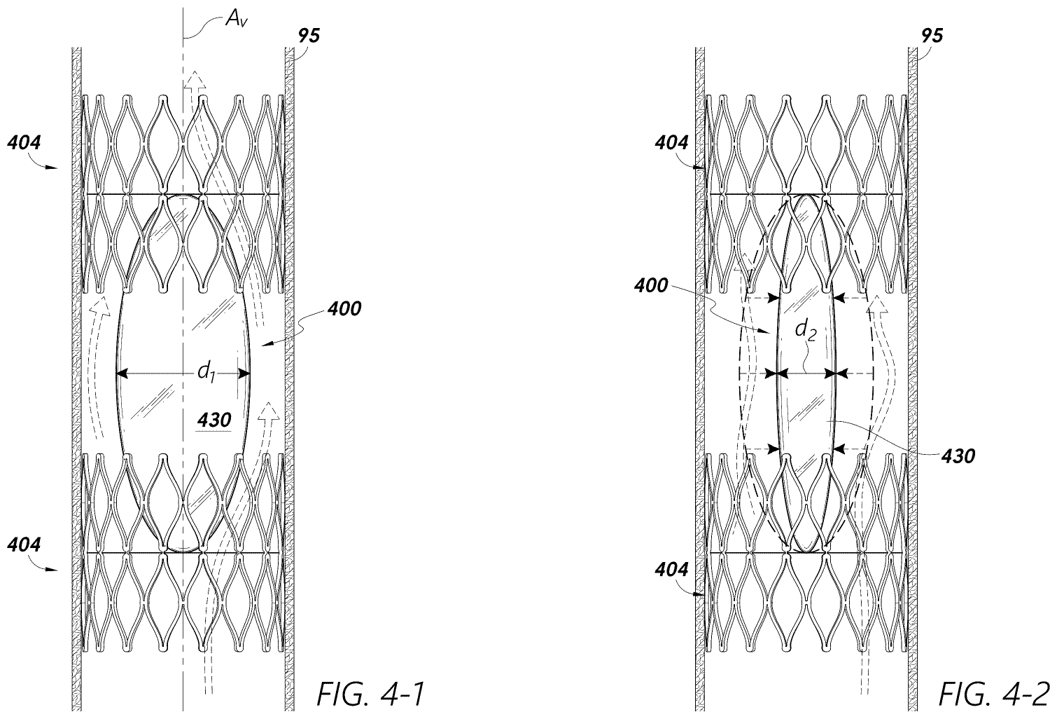

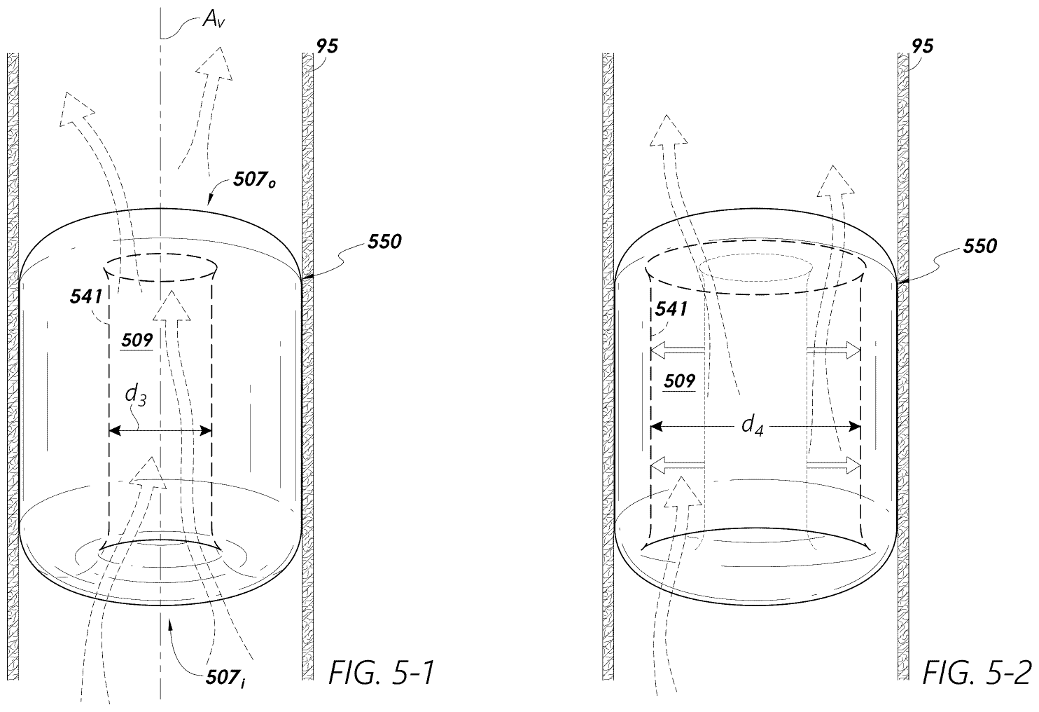

FIG. 3A shows an example stiff aorta 16′. FIGS. 3B and 3C show side and axial cross-sectional views, respectively, of the stiff aorta 16′ of FIG. 3A experiencing compromised expansion and contraction over a cardiac cycle.

As shown in FIG. 3A, the aorta tends to change in shape as a function of age, resulting in a higher degree of curvature and/or tortuosity over time. As the vasculature of a subject becomes less elastic, arterial blood pressure (e.g., left-ventricular afterload) becomes more pulsatile, which can have a deleterious effect. For example, undesirably pulsatile arterial blood flow, such as the thickening of the left ventricle muscle and/or diastolic heart failure. Stiffness in the aorta and/or other blood vessel(s) can occur due to an increase in collagen content and/or a corresponding decrease in elastin.

With the walls of the blood vessel 16′ being resistant to stretching due to the stiffness thereof, the expansion of the blood vessel diameter from the non-expanded diameter d₁′ to the expanded diameter d₂′ may be limited/reduced compared to the expansion of diameter of a healthy blood vessel. Although FIGS. 3B and 3C show a small amount of expansion and volume change Δv′ experienced by the blood vessel 16′, in some cases, a blood vessel may be sufficiently stiff that substantially no vessel expansion takes place in systole.

In view of the health complications that may be associated with reduced arterial compliance, as described above, it may be desirable in certain patients and/or under certain conditions, to at least partially alter compliance properties of the aorta or other artery or blood vessel, or otherwise alter/control flow therein, in order to improve cardiac and/or other organ health. Disclosed herein are various devices and methods for at least partially restoring compliance to a blood vessel, such as the aorta. Certain examples disclosed herein achieve restoration of arterial compliance through the use of implantable compliant fluid channels/tubes or other forms (e.g., spheroid forms), through or around which blood circulation may flow. For example, a compliance-restoration device in accordance with the present disclosure may comprise an expandable fluid channel that expands and stores energy during higher-pressure periods of the cardiac cycle (e.g., during the systolic phase) and contracts/compresses during lower-pressure period (e.g., during the diastolic phase) to return the stored energy to the circulation and increase flow through the channel. The expansion of such fluid channels can be enabled by the compression of case contained in chamber(s) radially outside of the channel.

Arterial compliance restoration devices, methods, and concepts disclosed herein may be generally described in the context of the ascending aorta. However, it should be understood that such devices, methods and/or concepts may be applicable in connection with any other artery or blood vessel. Generally, the majority of aortic compliance is provided in the ascending aorta 12 with respect to healthy anatomy. Furthermore, calcification frequently occurs in the area of the ascending aorta 12, near the aortic arch 13 and the great vessels emanating therefrom. Such anatomical areas can experience relatively higher stresses due to the geometry, elasticity, and flow dynamics associated therewith. Therefore, implantation/deployment of compliance-enhancing, compressible-gas-filled implant devices of the present disclosure can advantageously be in the ascending aorta 12 in some cases. While relatively less calcification tends to occur in the descending 14 and abdominal 15 aorta, implant devices of the present disclosure can advantageously be implanted/deployed in such areas as well for the purpose of increasing compliance in the aortic system. Examples of the present disclosure provide compliance-enhancing gas-filled implant devices, which may be implanted in one or more locations in a compromised aorta and/or other vessel(s). For example, FIG. 3A shows example positions of gas-filled implant devices 101 including features disclosed herein implanted in various areas of an aorta 16′.

In some examples, devices of the present disclosure include compressible fluid (e.g., gas) chamber(s) surrounding a compliant fluid channel of the device, wherein the chamber(s) may be disposed within an outer frame, which may be configured to dilate the blood vessel in the area where the implant is deployed. The channel may provide a volume through which blood in the vessel may flow to traverse the implant device. While some example implant devices of the present disclosure are configured as tubes, wherein blood axially traverses the implant device through a central axial flow channel, it should be understood that compressible-gas implant devices with liquid buffer chamber(s) in accordance with the present disclosure can be configured to have blood flow traverse the implant by flowing around, rather than through, the implant, at least in part. Such devices can be configured, for example, as spheroid or other-shaped balloons. With respect to either implementation, luminal fluid pressure in the target vessel in the area of the implant can cause compressible-gas-filled chamber(s) of the device to compress and expand in a cyclical manner as to increase compliance and/or diastolic flow in the blood vessel. Devices of the present disclosure can be secured in-place in the target blood vessel via one or more frame (e.g., stent frame) components, which may comprise metal or other at least partially rigid material. Such frames can be configured to expand within the target blood vessel to cause dilation thereof, wherein the dilation of the blood vessel can serve to both secure the frame in the desired position within the target blood vessel, and further to create a space to accommodate the volumes of the gas- and/or liquid-filled chamber(s) of the device.

Devices of the present disclosure may include additional anchoring features to provide secure retention in the target blood vessel. For example, barb-type anchors may be integrated with an anchor frame of the device. Certain coverings and/or linings (e.g., cloth, polymer) may be implemented on frame components of implant devices of the present disclosure to improve fluid-sealing characteristics of the implant device and/or promote in-growth with the native blood vessel tissue, Compliance restoration devices disclosed herein may serve to at least partially increase coronary perfusion.

As referenced above, heart failure with preserved ejection fraction can be associated with certain comorbidities. For example, hospitalization at one year is prevalent among hypertensive HFpEF stage 3/4 patients with pulse pressure exceeding 70 mmHg. Thus, solutions designed to add compliance and/or equivalent fluid dynamic effects to the arterial system to reduce pulsatile left ventricular afterload can improve patient outcomes.

### Compliance-Enhancing Implant Devices

In some instances, examples of the present disclosure comprise compressible-gas-filled sleeves configured for implantation within a relatively stiff portion of the aorta or other blood vessel, wherein such implants add/enhance compliance of/in the blood vessel in response to changing blood pressure in the vessel. Implant devices of the present disclosure can include double-walled compartments filled with compressible gas in one or more compartments thereof, as well as liquid (which may not be compressible) in one or more buffering chambers/compartments, wherein the liquid-filled compartment(s) serve as a barrier/buffer for gas diffusion into the bloodstream from the gas-filled compartment(s). Compression of gas-filled chambers of devices of the present disclosure during systole can store energy in the compressed gas, which can be returned to the circulation during diastole through expansion of the gas, thereby reducing systolic pressure and increasing diastolic pressure. By implementing compliance-enhancing implant devices as disclosed herein within a target blood vessel, as opposed to solutions involving blood vessel grafts and/or resection, incidences of blood leakage and/or rupture of the devices can be contained within the target blood vessel, thereby reducing hazards associated with extravascular arterial blood, liquid, and/or gas leakage, such as within the abdominal and/or chest cavity.

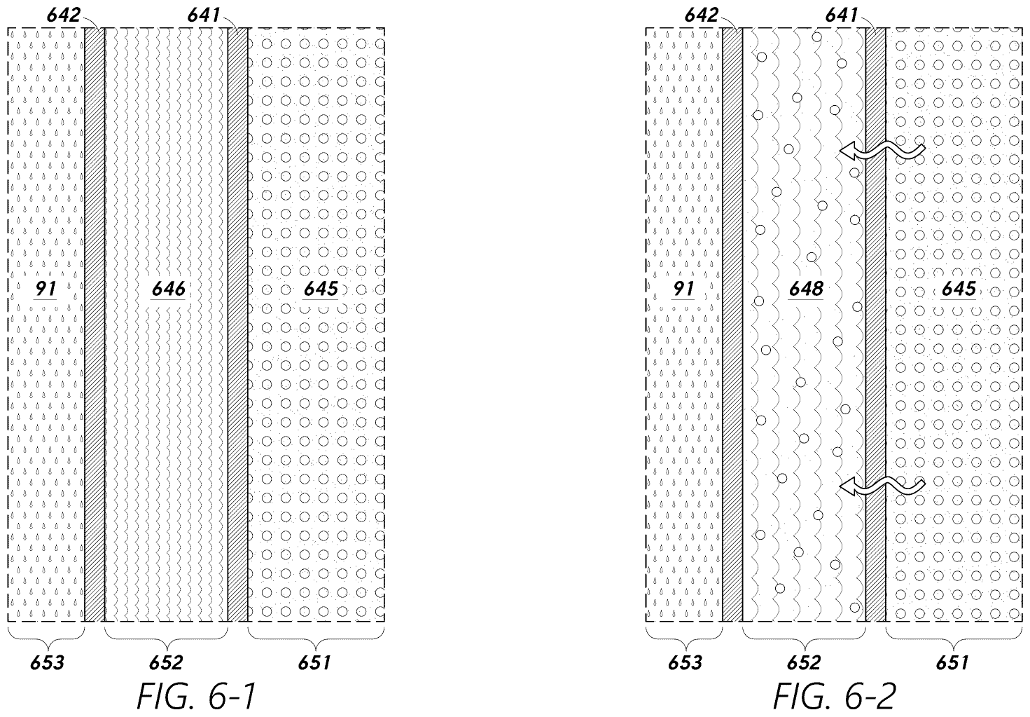

FIGS. 4-1 and 4-2 show an axially-oriented, compressible compliance-enhancing implant device 400 in expanded and compressed configurations, respectively, in accordance with one or more examples. The implant device 400 comprises a volume/form 430 configured to become compressed and/or reshaped in response to increasing fluid pressure in a blood vessel or other chamber 95 in which the implant 400 is implanted. For example, the implant 400 may include a spheroid, cylindrical, or other-shaped form/volume configured such that pressure against an outer surface thereof causes the device to compress and/or re-shape from a form having a first expanded diameter d₁ to a compressed diameter d₂ (shown in FIG. 4-2), wherein subsiding pressure conditions allow for and/or cause the device form to re-expand to the expanded diameter d₁. Such alternation between the expanded d₁ and compressed d₂ diameters can result in a change in volume occupied by the device 400 in correlation with changing pressure conditions, wherein such change in volume can reduce systemic pressure during high-pressure conditions (e.g., systole) and increase fluid pressure during lower-pressure conditions (e.g., diastole). Such effects on the systemic flow in the vessel 95 can increase compliance characteristics of the blood vessel 95 in the case where the compliance thereof has been compromised due to aging and/or other conditions. When compressed, as shown in FIG. 4-2, the device 400 may store energy associated with the biasing of the shape thereof in the expanded configuration shown in FIG. 4-1, such that such energy may be returned to the blood circulation as pressure subsides, thereby increasing flow therein.

The implant device 400 is shown as an example axial compliance-enhancing implant device, wherein the device 400 is aligned with and/or overlaps a central axis Av of the blood vessel 95, such that blood flow through the portion of the blood vessel 95 shown traverses/passes-through the blood vessel in the area around the spheroid form 400. Although shown and described as an axial implant device, it should be understood that devices similar to the device 400 may be implanted within a target blood vessel in a non-overlapping position with respect to the axis Av of the blood vessel 95. For example, the implant 400 may be positioned against an interior wall of the blood vessel, wherein blood flow through the vessel passes over and/or around at least a portion of the outside of the implant device 400. Any of the example compliance-enhancing implant devices of the present disclosure may be similar in one or more respects to the implant device 400, with respect to shape, configuration, components, and/or other aspects/features thereof.

The implant device form/structure 430 may be biased in the expanded state shown in FIG. 4-1 by gas or other media contained therein, wherein such media can be compressed under high pressures. Additionally or alternatively, the form 430 of the device 400 may be biased in the expanded configuration by mechanical attributes of the form/frame, which may hold the device 400 in the expanded configuration in the absence of sufficient external fluid pressure. For example, a wireframe or other structure may form the spheroid shape of the device 400, wherein the frame may be covered by a fluid-tight covering of the device 400. The device 400 may be vacuum-filled, or may comprise certain compressible media. The cover of the device 400 may or may not be elastic.

The implant 400 may be anchored within the target blood vessel 95 in any suitable or desirable manner. For example, in some implementations, as shown in FIGS. 4-1 and 4-2, one or more stents or other anchors 404 may be deployed within the blood vessel 95, wherein such anchors hold the implant 400 in the desired position within the blood vessel 95, such as within a central area/region within the vessel. The anchor(s) 404 may advantageously be designed to be deployed within the blood vessel 95 without substantially impeding the flow of blood around the implant device 400.

FIGS. 5-1 and 5-2 show a tubular compressible compliance-enhancing implant device 500 in expanded and compressed configurations, respectively, in accordance with one or more examples. Whereas the implant device 400 of FIGS. 4-1 and 4-2 is configured to be compressed in a manner such that fluid pressures outside of the device 400 cause inward compression of the structure thereof (with respect to an axis of the device 400 and/or the axis Av of the target blood vessel), other examples (e.g., the device 500) may be implemented in which outward expansion of an axial flow channel 509 of the device (with respect to an axis of the device 400 and/or the axis Av of the target blood vessel) causes compression of one or more chamber(s) of the device. For example, blood pressure/flow in the axial flow channel 509 can cause outward expansion of the flow channel 509, thereby increasing the volume of the flow channel 509 and compression/reducing a volume of the structure of the device radially outside of the flow channel 509. Such compression can store energy, wherein subsequent re-expansion of the volume of the structure of the device 550 radially outside of the flow channel 509 can return energy to the through-device circulation and thereby improve compliance/blood-flow characteristics.

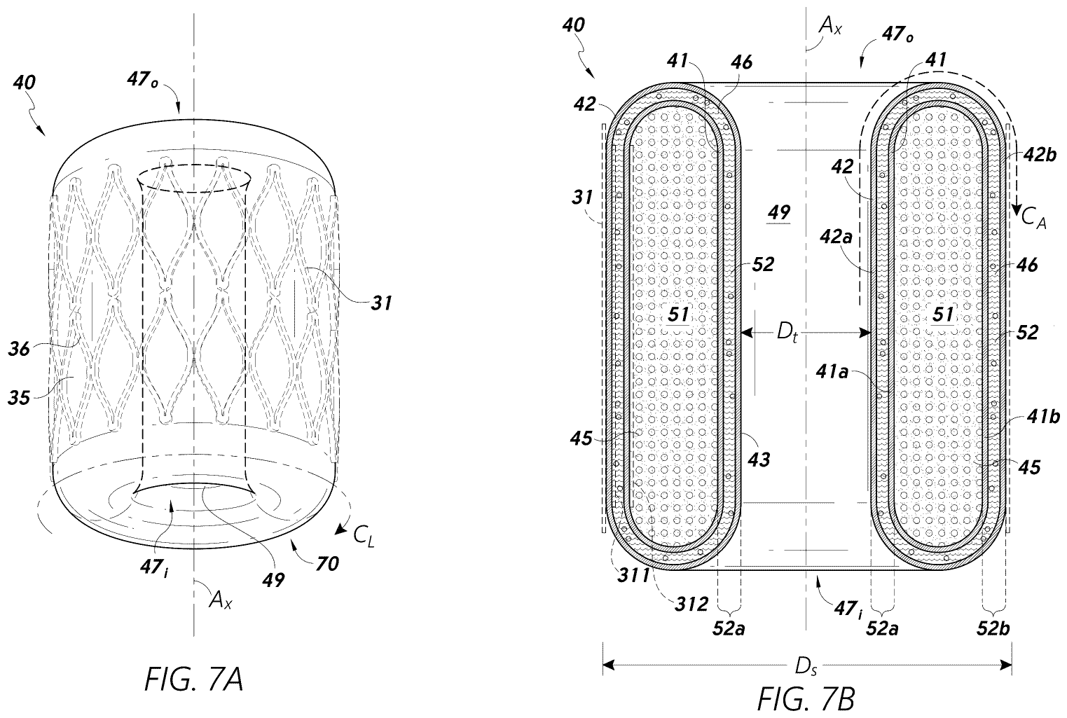

The shape of the device 550 may be considered a cylindrical toroid shape/form, wherein such form comprises a tubular balloon forming an flow channel 509. Blood flow may flow into the channel 509 through an inlet 507i and may pass out of the channel 509 through a downstream outlet 5070, as shown. High fluid pressure within the vessel 95 may result in similar pressure within the flow channel 509, which may cause the inner diameter/wall 541 to expand radially outward, thereby compressing the media/space disposed radially outside of the inner diameter/wall 541 within the device 550. When the wall 541 expands and/or stretches radially outward, the volume occupied by the device 550 within the vessel 95 decreases. Such compressed configuration is shown in FIG. 5-2, wherein the diameter of the flow channel 509 as expanded from the relaxed diameter da shown in FIG. 5-1 to the expanded diameter d₄ shown in FIG. 5-2. Compression of the tubular volume of the device 550 due to increased fluid pressure can result in energy being stored in the device 550, wherein reduction of fluid pressure in the channel 509 may allow the energy to be returned to the blood flowing therethrough due to the biasing of the channel 509 to the relaxed diameter/configuration shown in FIG. 5-1. As with other examples disclosed herein, the internal volume of the device 550 may be occupied at least in part by a vacuum or compressible media.

With respect to the implant devices 400 and 550 described above, where such implant devices contain gas, the walls of the device containing such gas media may comprise any suitable or desirable material known to those having ordinary skill in the art. However, such materials, which are advantageously biocompatible for permanent or temporary implantation of the device within the circulatory system (e.g., elastic materials, polymers, metals, and the like), can be prone to diffuse through the membrane into the surrounding environment (e.g., into the bloodstream with respect to intravascular implant devices) to some degree over time. For example, due to the relatively small molecular size of suitable gases for implantation (e.g., oxygen, carbon dioxide), out-gassing of the pressurized gas to the outside atmosphere through the cover/membrane in which the gas is contained can occur, which may be detrimental to the health of the patient in some instances. Furthermore, where such pressurized gases are relied upon for the purpose of storing and returning energy within the bloodstream to increase compliance therein, diffusion/out-gassing of the gas molecules from the compartment(s) in which they are contained can reduce the efficacy of the implant device by reducing the pressure and/or volume of the compressible compartment(s) of the implant device.

Embodiments of the present disclosure provide for gas-filled implant devices, wherein such gases are buffered from leakage into the bloodstream by layer(s) of liquid, wherein gas escaping the container thereof escapes into such liquid layer(s) rather than into the bloodstream. As liquids contained in the liquid buffer compartment(s) may not be prone to leak from the containers thereof, leakage of media contained within the device may be reduced or prevented relative to implant devices not including such buffer compartment(s)/layer(s),

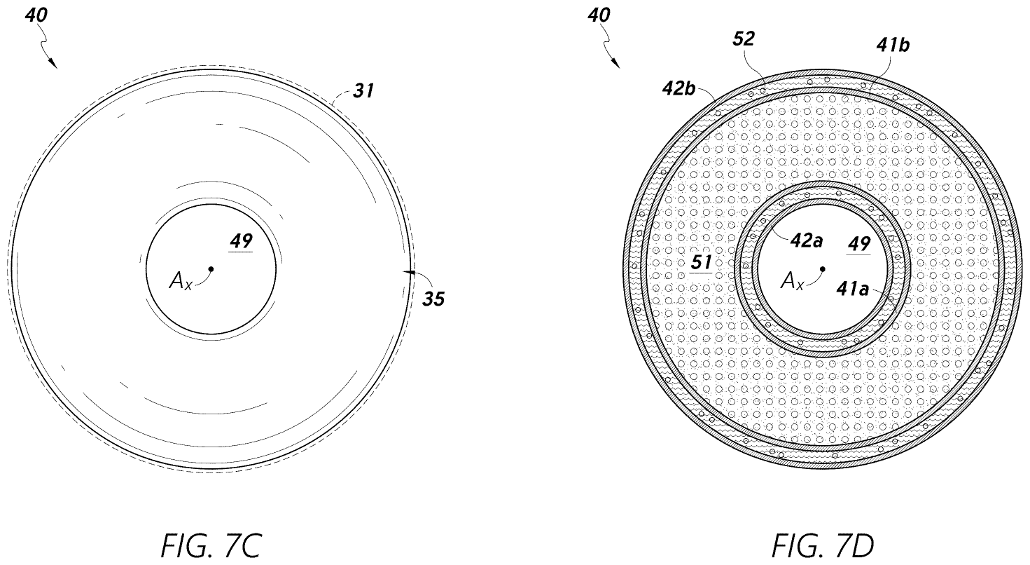

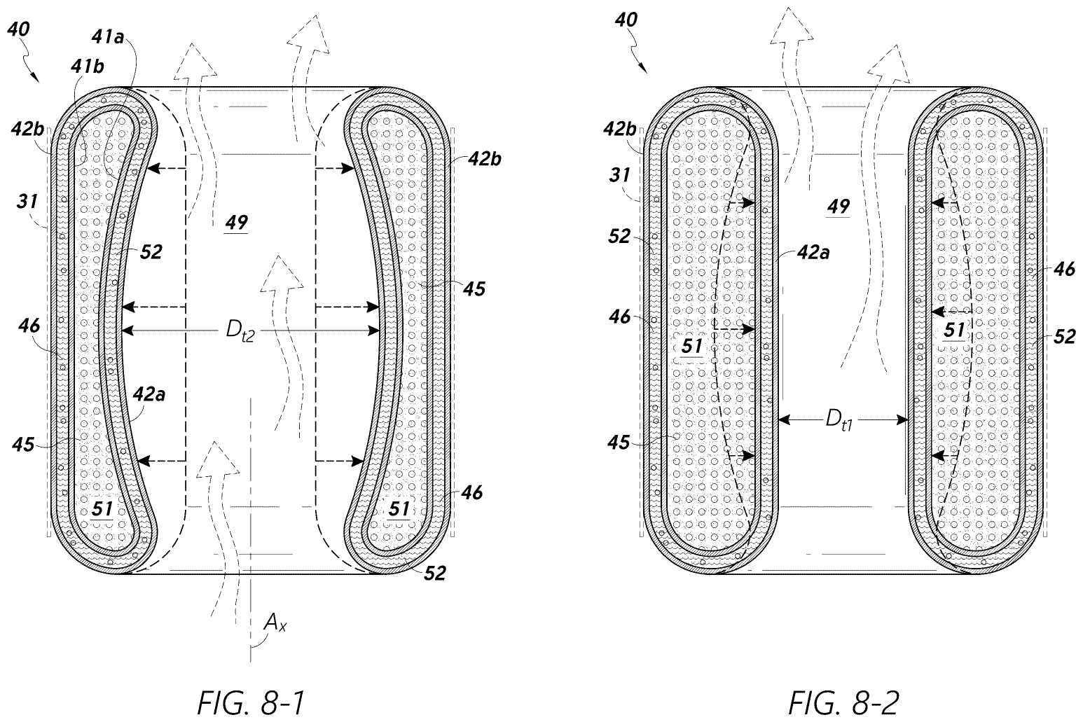

FIG. 6-1 shows a gas-filled chamber 651 and a liquid-filled chamber 652 separated by a membrane 641 in accordance with one or more examples. FIG. 6-2 shows the gas-filled chamber 651 and liquid-filled chamber 652 of FIG. 6-1 after some amount of diffusion of molecules from the gas-filled chamber 651 into the liquid-filled chamber 652 through the membrane 641 in accordance with one or more examples. As shown, a first layer, barrier, or membrane 641 may separate the gas-filled chamber 651 from the fluid-filled chamber 652, whereas another layer, barrier, or membrane 642 may separate the liquid-filled chamber 652 from the external environment 653. The outside environment 653 may be, for example, an interior of a blood vessel having blood 91 flowing/disposed therein. The gas-filled compartment/chamber 651 and the liquid-filled chamber/compartment 652 may be part of any compliance-enhancing implant device of the present disclosure. The two membranes 641, 642 may provide a double-wall configuration/solution for buffering the gas 645 contained in the gas-filled chamber 651 from the blood 91 outside of the implant device.

As described above, biocompatible gases contained within compartments/chambers of an implant device may be prone to outgassing/diffusion of gas molecules through the membrane(s) containing such media. In view of the potential negative impact of outgassing/diffusion of gas molecules into the bloodstream, examples of the present disclosure advantageously provide safer compliance-enhancing implant solutions through the use of liquid-filled buffer compartments. The effect of such liquid-filled buffering compartment(s) can be demonstrated with reference to FIGS. 6-1 and 6-2. For example, FIG. 6-1 shows gas 645 contained within the gas-filled compartment/chamber 651, wherein such gas 645 is pressurized and contained by the wall/membrane 641 associated therewith. That is, pressure of the gas 645 may exert a force against the membrane/wall 641, which may ultimately result in some amount of diffusion of the gas being pushed through the membrane, even in instances in which the membrane 641 is considered air-tight.

In FIG. 6-1, the gas 645 has not defused to a substantial degree through the membrane 641. However, over time, some amount of diffusion can be expected through the membrane. Therefore, the liquid-filled compartment/chamber 652, which contains certain liquid, gel, or other at least partially liquid medium 646 therein, may be implemented to prevent gas that diffuses through the membrane 641 from entering the bloodstream 91. For example, as shown in FIG. 6-2, gas diffusion through the membrane 641 may enter the liquid-filled chamber 652, where such gas molecules may interact with the liquid 646 in some manner. The gas 645 may comprise air, carbon dioxide, or other relatively large gas molecules, wherein such molecules may be prone to dissolve in a relatively benign manner in the fluid (e.g., saline solution) 646. Generally, the gas molecules that have defused into the liquid 646 may be less inclined to breach the outer area/membrane 642 into the bloodstream 91 compared to the tendency of the gas 645 to breach the membrane 641.

As gas molecules are introduced into the liquid 646, the liquid 646 may reach saturation with the gas; saturated liquid 648 is shown in shown in FIG. 6-2. The liquid 646 may comprise any suitable at least partially liquid medium, such as saline solution or the like. Once the liquid 648 has reached the saturation condition, wherein a maximum amount of the gas 645 has dissolved into the liquid 646, further diffusion of the gas 645 into the liquid-filled compartment 652 may cease due to the relative concentrations of the gas in the compartments 651, 652. Furthermore, once the gas 645 that has crossed the membrane 641 is dissolved into the liquid 646, it may become more difficult for such gas molecules to defuse out through the outer barrier/membrane 642, thereby preventing outgassing/diffusion into the bloodstream 91, Furthermore, as the liquid 646 may have sufficient molecular size to prevent the liquid 646 from breaching the outer wall/membrane 642 and leaking into the bloodstream, the outer barrier/membrane 642 may protect from any leakage of liquid or gas molecules into the bloodstream 91. That is, the ability of the membrane 642 to seal a liquid may be significantly greater than the ability thereof to seal a gas. Therefore, when the gas 645 has saturated the liquid 646, the membrane 642 may be sufficient to seal-off the implant device in a manner as to prevent diffusion through the membrane 642. Such may be the case even in implementations in which the outer membrane 642 comprises the same material as the inner membrane 641, which, as described above, may be prone to allow some amount of diffusion therethrough of the gas 645. It may be desirable to implement the gas 645 as carbon dioxide (CO₂) or similarly biocompatible gas, such that in case of rupture/failure of the membrane(s) 641, 642, potential leakage of the gases 45 into the bloodstream 91 to be relatively safe.

The mixture of the liquid 646 with the gas 645 in the liquid-filled compartment 652, once equilibrium/saturation is reached within the compartment 652, can prevent further leaking of the gas 645 through the membrane 641 into the compartment 652. Such prevention of further diffusion/leaking of the gas 645 can advantageously serve to maintain the fluid pressure within the gas-filled compartment 651 at a suitable level to support the efficacy of the compliance-enhancement characteristics of the implant device. Therefore, the configuration of the double-wall, buffered structure described in FIGS. 6-1 and 6-2, which may be implemented in connection with any of the examples of the present disclosure, can serve to reduce the amount of gas depletion over time due to gas diffusion, thereby reducing risks and improving efficacy of example implant devices of the present disclosure.

The liquid 646 may comprise any suitable or desirable liquid, which may advantageously be biocompatible to reduce risks associated with rupture of the membrane 642 resulting in leakage of the liquid 646 into the bloodstream 91. In some implementations, the liquid 646 is a relatively dense liquid, or gel. The buffer media described in connection with examples of the present disclosure may be any material that is at least partially liquid, and may be at least partially solid as well. For example, certain gel-type media/medium may be implemented in connection with some examples.

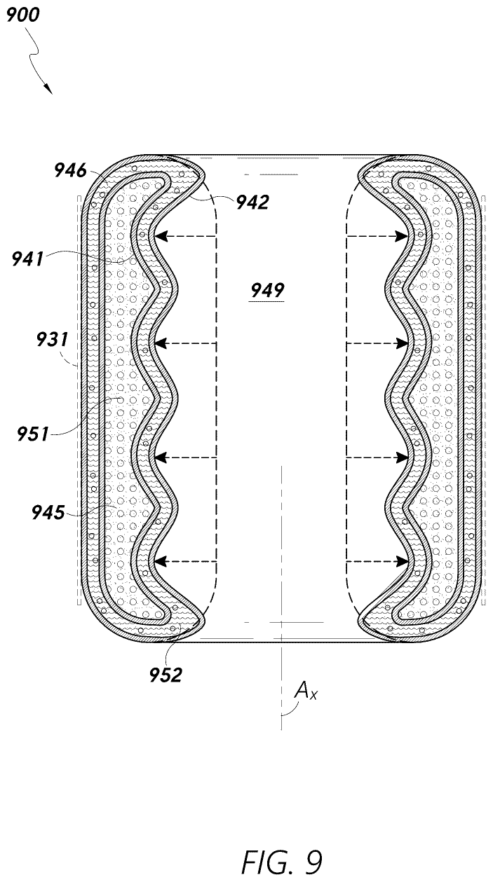

FIGS. 7A-7D provide views of a compliance-enhancing implant device 40 including one or more compressible-gas-filled chambers 51 buffered from the exterior of the device by one or more liquid-filled chambers 52 in accordance with one or more examples. The device 40 can include an anchor frame 31 and an inner tubular sleeve 70 coupled to or disposed within the anchor frame 31, wherein the tubular sleeve 70 forms a central axial channel 49. References herein to the device 40 can be understood to refer to the tubular sleeve 70 or collectively to the sleeve 70, the frame 31, and/or any other components associated with the device 40. In some examples, the device 40 does not include a frame. Furthermore, in some examples, the device 40 comprises a frame that is disposed at least partially within one of the chambers 51, 52.

The exterior of the device 40 may be considered the area within the flow channel 49 and any other area outside of the device 40. The device 40 has a tubular, cylindrical toroid shape forming an axial flow channel 49, wherein the body of the device comprises at least two fluid-sealed compartments, namely an internal gas-filled compartment 51 and an outer liquid-filled compartment 52, which buffers the gas-filled compartment 51 from the external environment. A membrane 41 divides the gas-filled compartment 51 from the liquid-filled compartment 52. Although the compartments 51, 52 are described in certain contexts as singular compartments, it should be understood that such terminology is used for convenience only, and any compartment or chamber described herein in the singular should be understood to refer to one or more compartments/chambers. In some implementations, one or both of the compartments 51, 52 can be partitioned into sub-compartments/chambers or other compartment/chamber portions.

The inner wall 43 of the tube/sleeve device 40 that forms the flow channel 49 may be radially-expandable, wherein radial expansion of the channel wall(s) 43 causes an increase in the volume of the channel 49 and a commensurate decrease in the volume of the gas-filled compartment/chamber 51 from compression of the gas 45 contained therein. As the inner channel-/tube-forming wall 43 radially expands and contracts, the volume of the channel 49 increases and decreases in a manner as to enhance compliance characteristics for the blood vessel in which the device 40 is implanted. The compliant channel 49 is defined/formed by the expandable inner wall(s) 43. The inner wall(s) 43 may further serve as a radially-inner diameter/boundary of the liquid-filled buffer compartment/chamber 52 and/or a portion thereof that is positioned radially inside of the gas compartment 51. The buffer compartment(s) 52 may envelope the gas compartment(s) 51.

The device 40 may include and/or be configured to be secured to an anchor frame 31, which may be positioned radially outside of toroid form, or within the gas chamber 51 (see dashed frame 312) or within the liquid-filled chamber 52 (see dashed frame 311). The frame 31 can be tubular in form with an axial channel therethrough. The frame 31 is configured to be expanded within a native blood vessel (e.g., aorta) to secure the implant 40 in place in the blood vessel.

The liquid-filled buffer chamber(s) 52 wraps/wrap axially around the gas-filled chamber(s) 51 radially inside and outside of the gas-filled chamber(s) 51. The liquid-filled chamber(s) 52 can further cover the axial ends of the gas-filled chamber(s) 51, as shown in FIG. 7C. The device 40 can have a cylindrical tube structure. The gas-filled portion 51 and the liquid-filled portion 52 can be considered a tubular balloon due to the fluid contained therein. The tubular balloon can be disposed within an expandable cylindrical frame. The interior 41 and exterior 42, 43 walls/membranes can comprise a common material.

The implant device 40 is configured to add back compliance to a target blood vessel in which it is implanted, such as the aorta, to improve perfusion of the heart muscle. The device 40 may be a percutaneously-/transcatheter-placeable implant configured to be compressed (e.g., radially compressed) and transported within a delivery catheter/sheath or other tubular delivery system. The radially-expandable inner wall/tube 43, in a natural, relaxed, and/or de-pressurized configuration/state can have a generally straight cylindrical shape/form, whereas in a radially-expanded, pressurized configuration/state, the wall/tube 43 may have an outwardly-/externally-convex (e.g., internally concave) cylindrical shape. In some implementations, as the inner wall/tube 43 is pressed/forced radially outward, thereby compressing the gas compartment 51, the channel 49 maintains a generally-cylindrical/tubular shape. The device 40 may advantageously function as an arterial flow optimizer to generate vascular compliance.

The anchor frame 31 may be an expandable stent-type frame configured to expand radially from a compressed delivery configuration to the expanded state shown. To achieve this change in the shape and dimension of the frame/stent 31, the frame 31 may have a structure comprising a plurality of struts 36 forming an array of cells 35, which may have any suitable or desirable shape (e.g., oval/ellipse, diamond/rhombus, hexagonal diamond/polygon, etc.). The cells of the frame 31 may be arranged in any number of columns in the circumferential direction and rows in the axial, or lengthwise, direction Aₓ. The cells of the frame 31 may be formed using any suitable process, such as by stamping or machining the frame structure from a sheet or tube of metal. The frame 31 may be made of any at least partially rigid material, such as metal or plastic. For example, the frame may comprise stainless steel or nitinol. Where nitinol or other shape-memory metal or material is implemented for the frame 31, the frame may be self-expanding. In some implementations, the array of struts of the frame 31 is formed from a sheet of metal, which is rolled into a cylinder to form the tubular/cylindrical form of the frame configured for placement within a blood vessel. In some implementations, a balloon catheter can be used to expand the frame 31 for securing in the wall of an artery or other blood vessel or body cavity.

The body of the device 40 can comprise a multilayer cylindrical toroid (e.g., axially-stretched-donut shape) balloon including at least the inner gas compartment 51 and internal 52a and external 52b liquid-filled compartment portions/layers, wherein the radially-inner 41a and radially-outer 41b walls of the gas compartment 51 are brought together, thereby compressing the gas 45, in response to a pressure gradient between the blood flowing through the axial channel 49 and the gas 45 disposed within the compartment(s)/chamber(s) 51. The device 40 may be implanted in, for example, the ascending aorta, such as in an area just above the aortic valve.

The body of the implant device 40 may have a sleeve-type shape, as illustrated. The device 40 may be implanted in a relatively stiff portion of the aorta or other target blood vessel. The device/sleeve 40 can include a double-walled compartment including a liquid-filled compartment 52 positioned around the internal gas-filled compartment 51, wherein the liquid 46 is provided in the compartment(s) 52 as a mechanism of providing a barrier for gas diffusion from the gas compartment(s) 51 and into the bloodstream within the channel 49 and/or otherwise external to the device 40.

The gas-filled expandable sleeve implant device 40 can comprise any suitable or desirable flexible materials that serve as the various walls/membranes defining the fluid-filled compartments thereof. The inner gas-filled compartment(s) 51, as described, are surrounded on one or more sides thereof by a double-walled arrangement of membrane layers having at least partially liquid media 46 disposed therein/therebetween. While the inner compartment 51 comprises compressible gas, such as carbon dioxide or the like, the outer compartment 52 surrounding the inner compartment 51 is filled with liquid, such as saline or other compatible liquid. The liquid-filled compartment 52 provides an isolation layer of liquid serving as a buffer/barrier between the gas compartment 51 and the external environment.

In some examples, the outside of the frame 31 is covered with a fabric or polymer cover. Such cover may be disposed on an outer surface or area of the frame 31, and/or may be disposed/applied to the inner diameter of the frame 31. For example, the layer(s) of covering may be disposed in some implementations between the outer frame 31 and the outer layer 42b of the buffer chamber 52. In some implementations, the cover comprises a cloth or polymer sleeve which may be at least partially elastic, or alternatively nonelastic. Covering may be applied over or within the frame 31 in any suitable or desirable manner, such as an electrical or mechanical spinning (e.g., rotary jet spinning, electrospinning, or similar) application process or other deposition process known to those having ordinary skill in the art.

The frame 31 may be a self-expandable and/or balloon-expandable stent. The diameter Dₛ of the frame 31 and/or device 40 may be sized to match the diameter of the native blood vessel in which the frame 31 is deployed (e.g., within 10-20% of the diameter of the native vessel). The frame 31 and/or device 40 may have a diameter slightly larger than the diameter of the target blood vessel in order to facilitate anchoring of the device within the blood vessel when deployed and expanded. Generally, the diameter of the ascending aorta may be less than about 2.1 cm. With regard to the abdominal aorta, the diameter may be less than about 3.0 cm. The device 40 may have a diameter that is greater than 2 cm, and/or greater than 3 cm. The body of the device 40 can be coupled to the frame 31 in any suitable or desirable manner, such as by suturing, bonding, or adhesive attachment to the frame 31.

The device 40 can have a cylindrical toroid shape, with the flow channel 49 running down an axis Aₓ of the tubular form of the device. Although shown as a cylindrical/elliptical tube/toroid, in some examples, the device 40 has the shape of a circular torus (e.g., donut-shaped). The device 40 can function as an arterial flow optimizer to generate vascular compliance, as described in detail herein. Although described as elastic in some contexts, it should be understood that the walls/layers 41, 42, 43 of the device 40 may be at least partially inelastic. For example, either or both of the inner 41 and outer 42/43 layers/membranes of the device 40 may be inelastic.

The chamber(s) 51 can be filled with compressible gas that can compress in the presence of elevated pressure levels (e.g., systolic aortic pressure) and can be prone to decompress and expand to a greater volume as pressure decreases (e.g., diastolic aortic pressure), wherein such expansion induces blood flow within channel 49 of the device 40 and thereby in the target blood vessel (e.g., aorta).

The flow conduit 49 formed by the compressible/expandable device 40 is configured to contract and expand as the heart beat cycles, mimicking the compliance of healthy vascular tissue. The pressurization of the gas 51 can bias the device 40 towards a certain shape and/or expansion state, such that the expansion of the gas 45 promotes a return to such state/shape after high blood pressure causes compression/deformation of the gas 45, thereby introducing/providing compliance to promote blood flow in the blood vessel (e.g., aorta), The implant device 40 can have a circular (e.g., torus) or oval/oblong longitudinal cross-sectional shape (see FIG. 7C; e.g., cylindrical/elliptical toroid, as shown in the relevant figures). The device 40 can be compressible for delivery within a catheter/sheath. The device 40 may be placed in the ascending aorta, such as just about the aortic valve, or in any other position in the aorta or inferior vena cava.

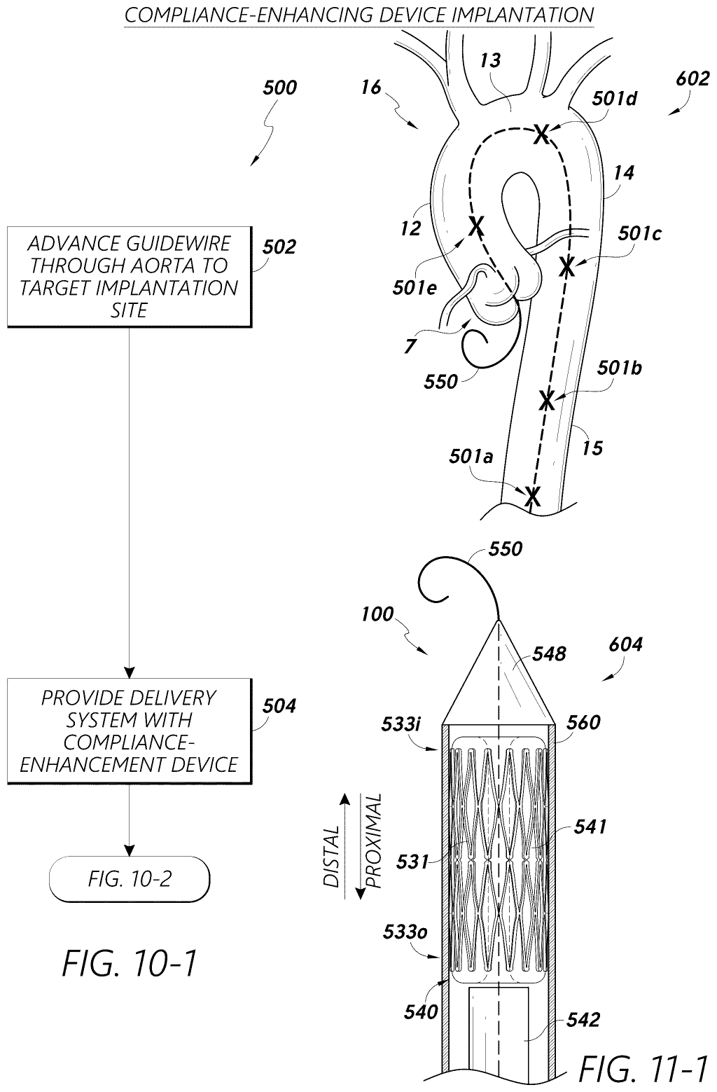

FIGS. 8-1 and 8-2 show side cross-sectional views of the compliance-enhancing implant device 40 of FIGS. 7A-7E in compressed and relaxed configurations, respectively, in accordance with one or more examples. In the image of FIG. 8-1, the flow channel 49 within the tubular device 40 is filled with blood/fluid having a higher fluid pressure than that of the gas medium 45 in the inner sealed chamber(s) 51. The gas-filled chamber(s) 51 is/are surrounded by the liquid-filled buffer chamber(s) 52, the inner wall/membrane 42a of which forms the flow channel 49. Therefore, the inner walls 41a, 42a of the chambers 51, 52 are deflected/expanded radially outward (relative to the axis Aₓ of the device 40) as the luminal pressure in the channel 49 exceeds and/or increases beyond a threshold level that is greater than the minimum diastolic pressure level, thereby compressing the gas 45 within the chamber(s) 51; the chamber(s) 52 may or may not be compressed. As the inner walls 41a, 42a are deflected radially-outward, the volume of the channel 49 increases and energy is stored in the device 40. For example, energy may be stored in the compressed gas 45 and/or in the material of the wall(s) 41a, 42b in the form of elastic stretch or other shape-memory features. The configuration/state of the implant device 40 in FIG. 8-1 may be associated with blood pressure conditions associated with the systolic phase of the cardiac cycle.

In the image of FIG. 8-2, the stored energy in the device 40 causes the inner walls 41a, 42a to contract/recoil back towards the original cylindrical tubular state thereof due to decreasing fluid pressure within the channel 49, thereby reducing the diameter Dₜ of the channel 49 from the expanded diameter Dₜ₂ to the relaxed diameter Dₜ₁. That is, as the pressure gradient between the channel 49 and the chamber(s) 51 decreases, the gas medium 45 within the chamber(s) 51 is permitted to expand, which returns the stored energy in the chamber(s) 51 and/or inner tube/layer(s) 41a, 42a to the blood circulation by decreasing the volume of the channel 49. The configuration/state of the implant device 40 in FIG. 8-2 may be associated with blood pressure conditions associated with the diastolic phase of the cardiac cycle.

The implant device 40 can be implanted in a relatively stiff portion of the aortic, or other blood vessel. For example, the implant device 40 may be deployed in an area of the ascending, descending and/or abdominal aorta, such that during systole, the gas 45 may be compressed within the internal compartment 51 and re-expand during diastole, thereby adding radial compliance in response to the blood pressure within the otherwise stiff aortic section.

The liquid-filled buffer chamber/layer 52 may serve to reduce risks associated with gas depletion over time, which can affect efficacy of a gas-pressure-reliant compliance implant device and/or can present health risk to the patient. The gas 45 and liquid 46, as with other gases and liquids disclosed herein, may be selected to allow for the gradual diffusion of the gas 45 into the liquid a 46 until an equilibrium concentration is reached. For example, the volume of the liquid 46 in the chamber 52 may be relatively limited. Therefore, the liquid 46 may become relatively quickly saturated by the gas 45. When the gas/liquid equilibrium/saturation is reached, the gas 45 will no longer diffuse into the liquid 46, or alternatively, diffusion may occur in both directions across the membrane 41 such that an equilibrium is achieved over time as a steady state. Generally, the rate of diffusion from gas to liquid is higher than diffusion rates from liquid to liquid, therefore gas 45 diffusing into the surrounding liquid 46 and the outer compartment 52 may tend to remain in the liquid 46 or diffuse back into the internal gas-filled chamber 51, rather than diffusing from the liquid chamber 52 into the external bloodstream. In some implementations, the particular gas and liquid are chosen such that once the gas 45 diffuses into the liquid 46, the molecules of both the gas and the liquid are chemically bonded to form a materially different liquid composition from the initial composition of the liquid 46, wherein the properties of the new liquid composition may prevent further diffusion of gas molecules therefrom through the outer membrane a 42 into the bloodstream.

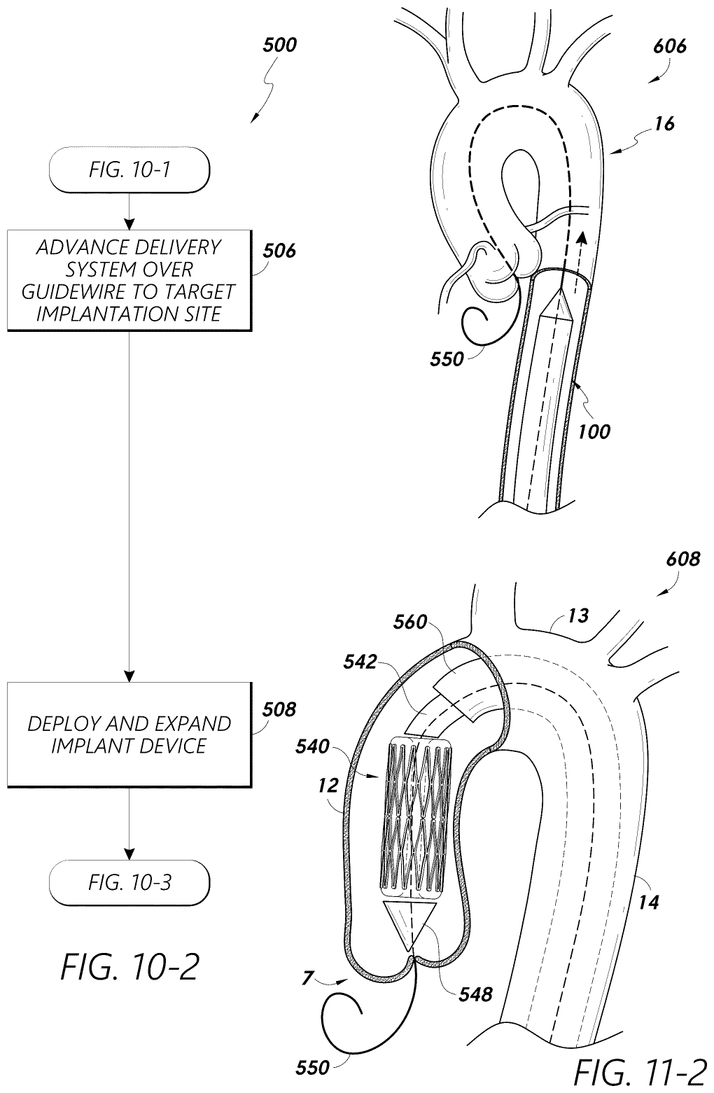

FIG. 9 shows a side cross-sectional view of a compliance-enhancing device 900 having non-elastic sidewalls in accordance with one or more examples. As described above, some examples of the present disclosure include elastic membranes/walls configured to stretch as the flow channel through the device expands radially in volume. In such examples, energy may be stored both in the compressed gas in the internal gas compartment(s)/chamber(s), as well as in the stretch of the membrane/walls of the device, such as the walls that define the flow channel. However, it should be understood that examples of the present disclosure may or may not be elastic with respect to the walls of the flow channel. For example, the device 900 of FIG. 9 includes inner channel-defining walls 942 that are flexible but inelastic. Therefore, high pressure within the channel 949 defined by the walls 942 causes radial outward deflection of the walls 942 without the walls 942 elastically stretching. Rather, the walls 942 may be inclined to deflect in an inelastic manner, as shown. Such inelastic deflection may result in certain folding or other flexing of the walls 942. In some implementations, the deflection of the walls 942 between low-pressure and high-pressure states may be limited by the area of the walls 942, wherein the walls 942 may be radially outwardly deflected until the area thereof is pulled tight and further deflection is constrained.

In addition to the inner channel-forming walls 942, which may provide a radially inner boundary/barrier for the liquid filled compartment(s) 952, the inner walls 942 separating the liquid 946 in the liquid-filled compartment 952 from the gas 945 in the gas-filled compartment 951 may or may not be elastic. For example, the outer 942 and inner 941 walls of the double-wall configuration shown may comprise the same material or different materials, each of which may or may not be elastic. The implant device 900 may include an anchor frame 931, which may be disposed on an outer radius/diameter of the device 900, or within one or more of the chambers of the device 900.

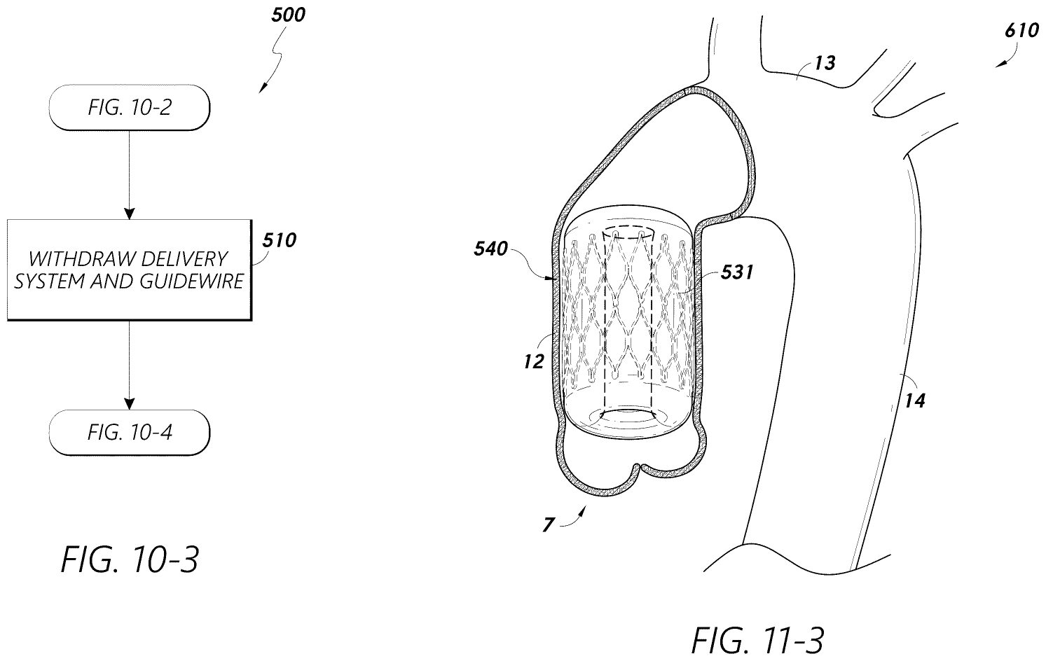

FIGS. 10-1, 10-2, 10-3, and 10-4 illustrate a flow diagram for a process 500 for implanting a compliance-enhancing implant device in accordance with one or more examples. FIGS. 11-1, 11-2, 11-3, and 11-4 provide images of the compliance implant device and certain anatomy corresponding to operations of the process 500 of FIGS. 10-1, 10-2, 10-3, and 10-4 according to one or more examples. The process 500 utilizes a transcatheter procedure for implantation/deployment of compliance-enhancement implant devices in accordance with aspects of the present disclosure. However, it should be understood that implant devices disclosed herein may be implanted using other types of minimally-invasive and/or surgical procedures.

At block 502, the process 500 involves advancing a guidewire 550 through at least a portion of the aorta 16 of the patient to reach a target implantation site 501. For example, image 602 shows an example implantation site 501a in the abdominal aorta 15, example implantation sites 501b, 501c in the descending thoracic aorta 14, an example implantation site 501d in the aortic arch 13, and an example implantation site 501e in the ascending aorta 12, such as in the area above the aortic valve 7. The guidewire 550 may be advanced through the aortic valve 7, or to any point along the path of the aorta 16. Access to the aorta 16 may be made through any suitable vessel puncture providing access to the arterial system. For example, access may be made via the femoral artery or other arterial blood vessel. In some implementations, access is made to the inferior vena cava via the femoral vein or other access, wherein a guidewire and/or other instrumentation may be crossed over into the abdominal aorta 15 in an area where the inferior vena cava and abdominal aorta 15 are adjacent to one another by puncturing through the venous wall and the arterial wall and advancing through such puncture openings. Although the process 500 and certain other examples are described herein in the context of implantation within the aorta 16, it should be understood that compliance-enhancement devices of the present disclosure may be implanted in other arterial or venous blood vessels, such as the inferior vena cava 19 (see FIG. 1).

Although the process 500 and accompanying illustrations are presented with respect to the implantation of a single compliance-enhancement implant device 540, it should be understood that the process 500 may involve implanting multiple compliance-enhancement implant devices in various positions within the aorta 16 or other blood vessel(s).

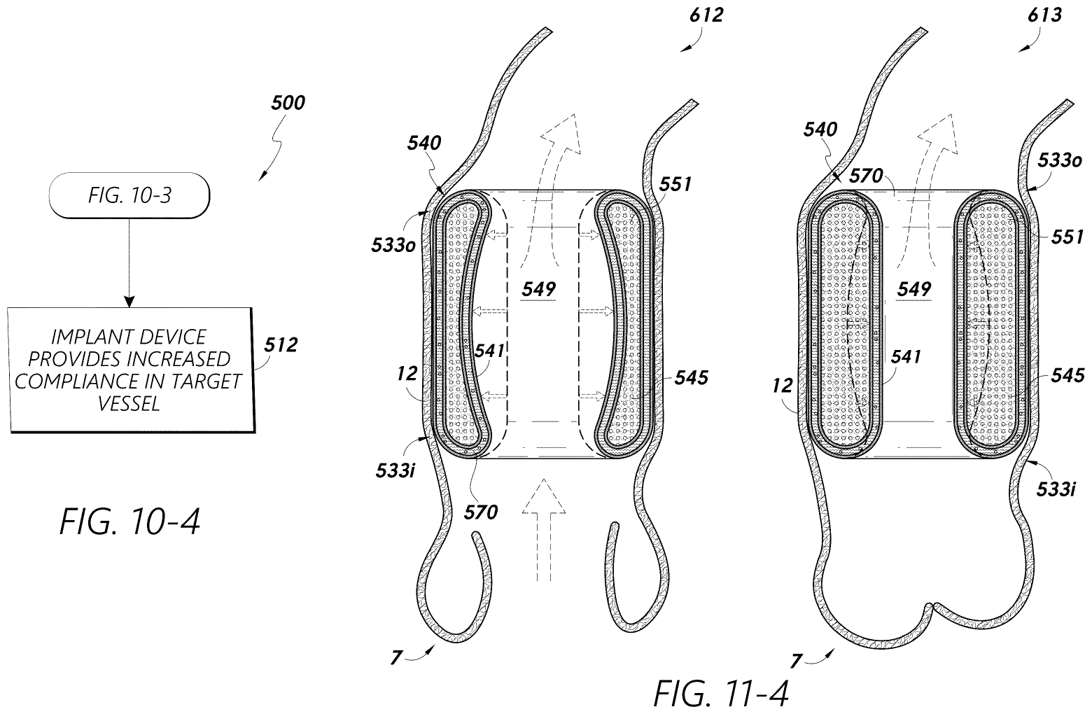

At block 504, the process 500 involves providing a delivery system 100 having a compliance-enhancement implant device 540 disposed in a distal portion thereof. Image 604 of FIG. 11-1 shows a cut-away view of an example implementation of the delivery system 100 in accordance with one or more examples of the present disclosure. The delivery system 100 can comprise one or more catheters or sheaths 560 used to advance and/or implant the compliance-enhancement implant device 540, which may be disposed at least partially within the delivery system 100 during portions of the process 500. The compliance-enhancement implant device 540 can be positioned within the delivery system 100 with a first end thereof (e.g., inflow end 533i) disposed distally and a second end (e.g., outflow end 5330) disposed proximally with respect to the illustrated orientation of the delivery system 100.

In some examples, the delivery system 100 comprises an outer catheter or shaft 560, which may be used to transport the compliance-enhancement implant device 540 to the target implantation site. That is, the compliance-enhancement implant device 540 may be advanced to the target implantation site at least partially within a lumen of the outer shaft 560, such that the compliance-enhancement implant device 540 is held and/or secured at least partially within a distal portion of the outer shaft 560 in a radially-compressed configuration.

In some examples, the delivery system 100 comprises a tapered nosecone feature 548, which may facilitate advancement of the distal end of the delivery system 100 through the tortuous anatomy of the patient and/or an outer delivery sheath or other conduit/path. The nosecone 548 may be a separate component from the outer shaft 560 or may be integrated with the outer shaft 560. In some examples, the nosecone 548 is distally tapered into a generally-conical shape and may comprise and/or be formed of multiple flap-type forms that can be urged/spread apart when the compliance-enhancement implant device 540 and/or any portions thereof, interior shafts, or devices, are advanced distally therethrough.

The delivery system 100 may further be configured to have the guidewire 550 disposed at least partially within the delivery system 100 and/or coupled thereto in a manner to allow the delivery system 100 to follow a path defined by the guidewire 550. In some implementations, the guidewire 550 may pass through an interior of the implant device 540 (e.g., through the flow channel of the device) and/or through a lumen of a pusher device or tube 542 of the delivery system 100.

The compliance-enhancement implant device 540 may have any of the features of any one or more of the examples described in detail herein, including an outer frame 531 and an inner, double-walled, gas- and liquid-containing sleeve. The implant device 540 may be disposed within the shaft/sheath 560 in a radially-compressed configuration, wherein the frame 531 and/or tube sleeve 540 is/are crimped to assume a reduce radial profile. In the compressed delivery configuration, the device 540 may be somewhat elongated compared to a fully-expanded configuration thereof due to at least some of the struts/cells of the frame 531 being deflected into more longitudinally-oriented configurations when radially crimped/compressed.

The delivery system 100 may optionally comprise the illustrated pusher shaft 542, which may be slidingly disposed within the outer sheath 560 proximal and/or adjacent to the implant device 540. The pusher 542 can be configured to be used to push/advance the frame 531 and/or other component(s) of the implant device 540 relative to the outer shaft/sheath 560 as a means to deploy the device 540 from the sheath 560. For example, the pusher 542 may be distally advanced relative to the outer sheath 560 to cause distal advancement of the compliance-enhancement implant device 540 through a distal opening in the outer sheath/shaft 560. Alternatively (or additionally), the implant device 540 may be deployed from the outer sheath 560 at least in part by proximally pulling the outer sheath 560 relative to the pusher 542.

At block 506, the process 500 involves advancing the delivery system 100 over the guidewire 550 until the target implantation site is reached to thereby position the implant device 540 for deployment in the target anatomy. At block 508, the process 500 involves deploying the implant device 540 from the delivery system 100, as shown in image 608, which may be performed in any of the manners described above. Once the device 540 is deployed from the delivery system 100, the process 500 may involve expanding the frame 531 and/or tube sleeve of the implant device 540 to thereby secure the implant 540 in place in the deployed/expanded configuration thereof.

The device 540 may be expanded by mounting the inner flow channel on a balloon of the delivery system 100. The device 540 can be situated on the balloon such that the inflow end 533i is disposed distally on the balloon and the outflow end 5330 is disposed proximally on the balloon 606. When the balloon is inflated, the frame 531 expands around the balloon and tube/sleeve of the device 540. The balloon may serve to expand the struts of the frame 531 by expanding the inner tube/sleeve 570 within the frame 531 to push outwardly against the frame 531, which may cause the gas in the gas-filled chamber(s) of the device 540 to compress to some degree during expansion of the balloon, but also exert outward radial force on the frame 531 to cause expansion thereof. A layer of coating, cloth, or other covering/layer may be disposed between the inner tube/sleeve 540 and the frame 531, wherein such layer may line the inner diameter of the frame 531. In some implementations, expansion of the frame 531 may be achieved via shape memory features of the frame 531. For example, the frame 531 may comprise nitinol or other shape-memory metal configured to self-expand when released from the delivery sheath/capsule.

At block 510, the process 500 involves withdrawing the delivery system 100 and guidewire 550, leaving the implant device 540 implanted in the aorta or other target blood vessel, as shown in image 610. At block 512, the process 500 involves maintaining the implant device 540 in the target blood vessel to thereby provide increased compliance in the target blood vessel. With the implant device 540 implanted, as shown, the increasing compliance provided by the implant device 540 can improve arterial blood flow and/or prevent elevated blood pressure. Other benefits may also be achieved, as described in detail herein.

As shown in image 612, when blood is received within the channel 549 that has pressure that is greater than the pressure in the gas-filled chamber(s) 551 of the tube 570, the inner wall 541 defining the channel 549 may radially deflect, thereby compressing the gas 545 due to the opposing force of the frame 531. Such gas compression results in stored energy in the gas 545. That is, the expansion energy of the compressed gas 545 disposed in the chamber 551 can increase in response to the outward radial deflection of the channel wall(s) 541. The outward deflection of the walls 541 serves to at least partially increase the volume of, and reduce the pressure in, the channel 549 during the high-pressure phase. When the pressure within the channel 549 reduces relative to the pressure of the gas 545 in the chamber 551, the stored energy in the compressed gas 545 may be returned to the blood circulation by reducing the volume of the channel 549 and increasing to some degree the pressure therein, thereby pushing blood through the channel 549 and out the outflow end 5330 of the device 540. Such volume reduction and pressure increase occurs in response to recoil/contraction of the tube 541 to the cylindrical shape/form shown in image 613.

Various examples of the present disclosure are disclosed in the context of compliance-enhancing implant devices implanted in arterial blood vessels. However, it should be understood that implant devices disclosed herein may be implanted in the venous system as well to improve compliance and/or otherwise control blood flow in target venous blood vessels (e.g., inferior vena cava). Furthermore, implant devices of the present disclosure may be utilized to increase pulmonary arterial compliance, which may be desirable for patients suffering from pulmonary hypertension.

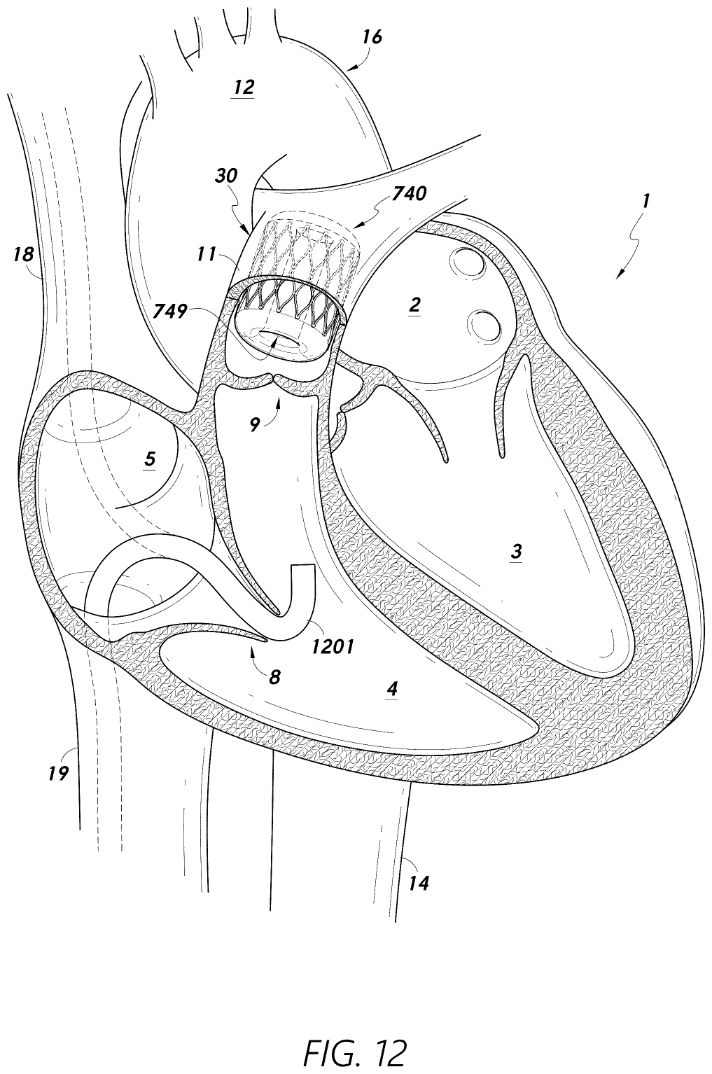

FIG. 12 shows a compliance-enhancing implant device 740 deployed in a pulmonary artery 11 of a heart 1 in accordance with one or more examples. Pulmonary circulation in healthy patients is typically a relatively low-pressure, high-compliance system. However, pulmonary arterial compliance can decrease in the presence of pulmonary hypertension due to increased extracellular collagen deposition in the pulmonary arteries. Loss of pulmonary arterial compliance can cause detrimental effects in patients with pulmonary hypertension due to the premature reflection of waves from the distal pulmonary vasculature, leading to increased pulsatile right ventricular afterload, which can contribute to right ventricular failure. Therefore, the implant device 740, by adding compliance to the pulmonary artery 11, can improve patient outcomes in some cases. Pulmonary arterial pressure in the flow channel 749 of the device 740 can cause gas in gas-filled chamber(s) of the device, as described in detail herein (see, e.g., FIG. 7B) to compress, thereby storing energy and returning the energy to the blood flow in the channel 749 as pressure subsides, allowing for expansion of the gas/device, as described in detail herein. The gas chamber(s) is/are buffered by liquid-filled chamber(s), as described in detail herein, thereby preventing out-gassing from the gas chamber(s) into the blood stream.

The implant device 740 may be advanced to the pulmonary artery 11 through a transcatheter access. For example, a delivery sheath/catheter 1201 may be advanced through the venous system, such as through the inferior 19 or superior 18 vena cava, into the right atrium 5, through the tricuspid valve 8, into the right ventricle 4, and into the pulmonary artery 11 through the pulmonary valve 9. Alternatively, the device 740 may be implanted using an open-chest surgical procedure.

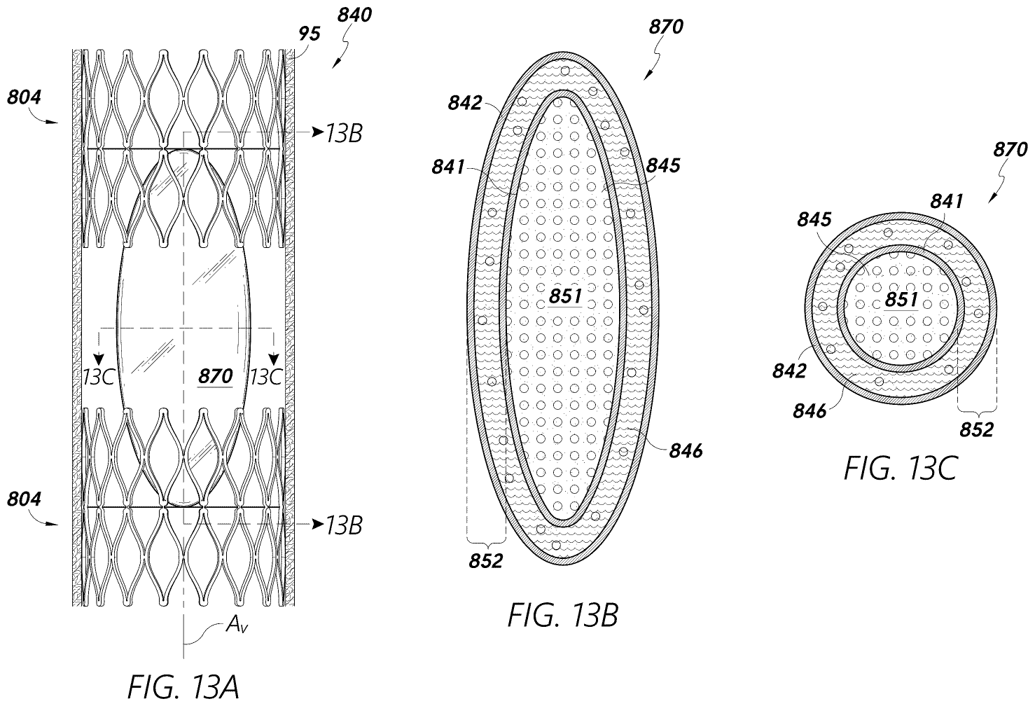

FIGS. 13A, 13B, and 13C provide side, side cross-sectional, and axial cross-sectional views, respectively, of an axial compliance-enhancing implant device 840 including a compressible-gas-filled chamber 851 and a liquid-filled buffer chamber/layer 852 in accordance with one or more example.

As described in detail above, some examples of the present disclosure of compliance-enhancing devices comprise tubular/sleeve devices having internal gas-filled chambers and liquid-filled buffer chambers/layers. However, it should be understood that compliance-enhancing devices comprising compressible-gas-filled chambers and liquid-filled buffer chambers in accordance with aspects of the present disclosure can have any suitable or desirable shape or form. The device 840 includes a balloon form 870, which may have any suitable or desirable shape, including the illustrated spheroid, blimp-type shape/form.

The balloon 870 includes an interior gas-filled chamber 851, which may be disposed at an axial center of a spheroid structure/form of the balloon 870 and has compressible gas 845 contained therein. The gas-filled chamber 851 is defined by a boundary comprising a membrane/wall 841, which may comprise any suitable or desirable materials as disclosed herein. The membrane 841 may be considered fluid-tight. However, the membrane/layer 841 may nevertheless, over time, be prone to permit some amount of diffusion of the gas 845 through the membrane 841. The buffer layer/chamber 852 is disposed/positioned around/about the chamber 851 in a manner as to buffer the gas chamber 851 from the external environment/atmosphere. Therefore, out-gassing of the gas 845 through the membrane 841 may collect in the liquid-filled layer/chamber 852 and combine in some manner with the liquid 846 contained therein. When the external pressure exceeds a pressure threshold that is greater than the minimum diastolic pressure, the liquid-filled buffer chamber 852 to deflect inwardly and press against the gas-filled chamber 851, thereby compressing the chamber 851.

The illustrations of FIGS. 13B and 13C show the implant device 840 in a state in which some amount of the gas 845 has diffused through the membrane 841 into the liquid-filled chamber 852. For example, state of the device 840 may represent a saturation state, in which a maximum amount of the gas 845 has diffused into the liquid 846 to produce a steady-state saturated medium comprising the liquid 846 and some amount of the gas 845, wherein the gas molecules 845 may or may not be chemically/molecularly bonded to the molecules of the liquid 846 in the saturated state.

The implant device 840 is shown in FIG. 13A as being implanted within a target blood vessel 95. When implanted, the balloon portion 870 of the device 840 may be anchored to the target blood vessel 95 in any suitable or desirable way, such as through the use of one or more stents 804, sutures, or other anchoring/attachment means or mechanism 804. FIG. 13A shows the balloon portion 870 coupled to one or more stent anchors 804, which may be expanded to be secured in place within the blood vessel 95. The anchoring means 804 may be configured to hold the balloon portion 870 generally overlapping with the axis Aᵥ of the blood vessel in coaxial alignment with the blood vessel, as illustrated. Alternatively, the anchoring means 804 may be configured to hold the balloon component 870 in any other position within the blood vessel 95 that is not overlapping with the axis Aᵥ thereof.

The liquid-filled buffer chamber/layer 852 may serve to reduce risks associated with gas depletion over time, which can affect efficacy of a gas-pressure-reliant compliance implant device and/or can present health risk to the patient. The gas 845 and liquid 846, as with other gases and liquids disclosed herein, may be selected to allow for the gradual diffusion of the gas into the liquid 846 until an equilibrium concentration is reached. For example, the volume of the liquid 846 and the chamber 852 may be relatively limited. Therefore, the liquid 846 may become relatively quickly saturated by the gas 845. When the gas/liquid equilibrium/saturation is reached, the gas 845 will no longer diffuse into the liquid 846, or alternatively, diffusion may occur in both directions across the membrane 841 such that an equilibrium is achieved over time. Generally, the rate of diffusion from gas to liquid is higher than diffusion rates from liquid to liquid. Therefore, gas diffusing into the surrounding liquid 846 and the outer compartment 852 may tend to remain in the liquid 846 or diffuse back into the internal gas-filled chamber 851, rather than diffusing from the liquid chamber 852 into the external bloodstream. In some implementations, the particular gas and liquid are chosen such that once the gas 845 diffuses into the liquid 846, the molecules of both the gas and the liquid are chemically bonded to form a materially different liquid composition from the initial composition of the liquid 846, wherein the properties of the new liquid composition may prevent further diffusion of gas molecules therefrom through the outer membrane 842 into the bloodstream.

### Additional Description of Examples

Provided below is a list of examples, each of which may include aspects of any of the other examples disclosed herein. Furthermore, aspects of any example described above may be implemented in any of the numbered examples provided below.

Example 1: An implant device comprising a first chamber containing a compressible gas medium, and a second chamber separated from the first chamber by a first membrane portion, the second chamber containing an at least partially liquid medium.

Example 2: The implant device of any example herein, in particular example 1, wherein the implant device is formed as a tubular structure having a central axial flow channel.

Example 3: The implant device of any example herein, in particular example 2, wherein the second chamber is separated from the flow channel by a second membrane portion.

Example 4: The implant device of any example herein, in particular example 3, wherein the first membrane portion and the second membrane portion are elastic.

Example 5: The implant device of any of any example herein, in particular examples 1-4 wherein the second chamber buffers the first chamber from an exterior of the implant device.

Example 6: The implant device of any example herein, in particular example 1, wherein the at least partially liquid medium is saturated with molecules of the compressible gas medium.

Example 7: The implant device of any example herein, in particular example 6, wherein the molecules of the compressible gas medium have diffused into the second chamber from the first chamber through the first membrane portion.

Example 8: The implant device of any example herein, in particular any of examples 1-7, further comprising an expandable frame.

Example 9: The implant device of any example herein, in particular example 8, wherein the frame is disposed radially outside of the first chamber and the second chamber with respect to an axis of the implant device.

Example 10: The implant device of any example herein, in particular example 8, wherein the frame is disposed at least partially within the first chamber.

Example 11: The implant device of any example herein, in particular example 8, wherein the frame is disposed at least partially within the second chamber.

Example 12: The implant device of any example herein, in particular any of examples 1-11, wherein the compressible gas medium comprises carbon dioxide.

Example 13: The implant device of any example herein, in particular any of examples 1-12, wherein the at least partially liquid medium comprises saline.

Example 14: The implant device of any example herein, in particular any of examples 1-13, wherein the implant device is formed as a spheroid structure.

Example 15: The implant device of any example herein, in particular example 14, wherein the first chamber is disposed at an axial center of the spheroid structure, and the second chamber is disposed radially outside of the first chamber.

Example 16: The implant device of any example herein, in particular example 14, further comprising one or more anchors configured to anchor the spheroid structure to a blood vessel.

Example 17: The implant device of any example herein, in particular example 16, wherein the one or more anchors are configured to hold the spheroid structure in a coaxial alignment with the blood vessel.

Example 18: The implant device of any example herein, in particular example 16, wherein the one or more anchors comprise first and second stents coupled to first and second axial ends, respectively, of the spheroid structure.

Example 19: An implant device comprising a stent frame, and a tubular balloon coupled to the stent frame, the tubular balloon forming a flow channel therethrough and including one or more gas-filled compartments, and one or more liquid-filled compartments that buffer the one or more gas-filled compartments from the flow channel.

Example 20: The implant device of any example herein, in particular example 19, wherein the one or more gas-filled compartments consists of a single gas-filled compartment.

Example 21: The implant device of any example herein, in particular example 20, wherein the one or more liquid-filled compartments consists of a single liquid-filled compartment.

Example 22: The implant device of any example herein, in particular any of example 19-21, wherein the one or more liquid-filled compartments wrap radially inside and radially outside of the one or more gas-filled compartments with respect to an axial dimension of the tubular balloon.

Example 23: The implant device of any example herein, in particular example 22, wherein the one or more liquid-filled compartments cover first and second axial ends of the one or more gas-filled compartments.

Example 24: The implant device of any example herein, in particular any of examples 19-23, wherein the one or more gas-filled compartments contain a compressible gas.

Example 25: The implant device of any example herein, in particular example 24, wherein the one or more liquid-filled compartments contain a liquid that is has molecules of the compressible gas diffused therein.

Example 26: The implant device of any example herein, in particular example 25, wherein the liquid is saturated with molecules of the compressible gas.

Example 27: The implant device of any example herein, in particular any of examples 19-26, wherein the tubular balloon comprises a double-walled structure including a first wall dividing the one or more liquid-filled compartments, and a second wall that serves as a barrier between the one or more liquid-filled compartments and an exterior of the tubular balloon.

Example 28: The implant device of any example herein, in particular example 27, wherein the second wall defines a flow channel that runs axially through the tubular balloon, and a cylindrical outer wall.

Example 29: The implant device of any example herein, in particular example 28, wherein the stent frame is disposed outside of the cylindrical outer wall.

Example 30: The implant device of any example herein, in particular example 27, wherein the first wall and the second wall comprise a common material.

Example 31: The implant device of any example herein, in particular example 27, wherein the first wall and the second wall are elastic.

Example 32: The implant device of any example herein, in particular any of examples 19-31, wherein the tubular balloon is attached to the stent frame.

Example 33: The implant device of any example herein, in particular any of examples 19-32, wherein the stent frame is disposed within at least one of the one or more gas-filled compartments.

Example 34: The implant device of any of any example herein, in particular examples 19-33, wherein the stent frame is disposed within at least one of the one or more liquid-filled compartments.

Example 35: The implant device of any of any example herein, in particular examples 19-34, wherein presence of fluid within the flow channel that is greater than a threshold level causes the flow channel to expand radially in volume, thereby compressing the one or more gas-filled compartments.

Example 36: A method of controlling blood flow in a blood vessel, the method comprising providing a delivery system having disposed therein an implant device, the implant device comprising a tubular frame in a radially-crimped configuration, and a cylindrical toroid structure disposed within the frame, the cylindrical toroid structure comprising a compressible-gas-filled chamber and a liquid-filled buffer chamber, advancing a distal portion of the delivery system to a target position within a portion of an aorta of a patient, deploying the implant device from a distal portion of the delivery system, and radially expanding the frame to secure the implant device at the target position within the aorta.

Example 37: The method of any example herein, in particular example 36, further comprising withdrawing the delivery system from the aorta, and receiving blood flow within a flow channel defined by an inner tubular wall of the cylindrical toroid structure, said blood flow causing the inner tubular wall to deflect radially outward, thereby causing the compressible-gas-filled chamber to compress and increasing a volume of the flow channel.

Example 38: The method of any example herein, in particular example 37, wherein, after said compression of the compressible-gas-filled chamber, the compressible-gas-filled chamber re-expands, thereby reducing the volume of the flow channel and pushing blood through the implant device.

Example 39: The method of any example herein, in particular example 37 or example 38, wherein said outward radial deflection of the inner tubular wall causes a first portion of the liquid-filled buffer chamber to press against the compressible-gas-filled chamber, thereby compressing the compressible-gas-filled chamber.

Example 40: The method of any example herein, in particular example 37 or example 38, wherein a first portion of the liquid-filled buffer chamber comprises a first portion disposed radially inside of the compressible-gas-filled chamber and a second portion disposed radially outside of the compressible-gas-filled chamber.

Example 41: The method of any example herein, in particular example 40, wherein the first and second portions of the liquid-filled buffers chamber are partitioned compartments of the liquid-filled buffer chamber.

Example 42: The method of any example herein, in particular any of examples 36-41, further comprising diffusing gas from the compressible-gas-filled chamber into the liquid-filled buffer chamber through a membrane.

Example 43: The method of any example herein, in particular example 42, wherein said diffusing the gas involves saturating liquid disposed in the liquid-filled buffer chamber with the gas.

Example 44: The method of any example herein, in particular any of examples 36-43, wherein the liquid-filled buffer chamber contains a gel medium.

Example 45: A method of controlling blood flow in a blood vessel, the method comprising providing a delivery system having disposed therein an implant device, the implant device comprising a spheroid balloon comprising an inner compressible-gas-filled chamber and an outer liquid-filled buffer chamber, advancing a distal portion of the delivery system to a target position within a portion of an aorta of a patient, deploying the implant device from a distal portion of the delivery system, and anchoring the implant device to the blood vessel at the target position within the aorta.

Example 46: The method of any example herein, in particular example 45, further comprising withdrawing the delivery system from the aorta, and permitting blood flow around an outer surface of the spheroid balloon, said blood flow causing the compressible-gas-filled chamber to compress and decrease a volume of the spheroid balloon.

Example 47: The method of any example herein, in particular example 46, wherein, after said compression of the compressible-gas-filled chamber, the compressible-gas-filled chamber re-expands, thereby increasing the volume of the spheroid balloon and pushing blood through the target position in the aorta.

Example 48: The method of any example herein, in particular example 46 or example 47, wherein said blood flow around the outer surface of the spheroid balloon exerts force against the outer surface that causes the liquid-filled buffer chamber to deflect inwardly and press against the compressible-gas-filled chamber, thereby compressing the compressible-gas-filled chamber.

Example 49: The method of any example herein, in particular any of examples 45-47, further comprising diffusing gas from the compressible-gas-filled chamber into the liquid-filled buffer chamber through a membrane that separates the compressible-gas-filled chamber from the liquid-filled buffer chamber.

Example 50: The method of any example herein, in particular example 49, wherein said diffusing the gas involves saturating liquid disposed in the liquid-filled buffer chamber with the gas.

Example 51: The method of any example herein, in particular any of examples 45-50, wherein the liquid-filled buffer chamber contains at least one of saline or a gel medium.

In any of the examples 1-51, the implant device can be sterilized.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A compliance-enhancing implant device dimensioned for deployment within a blood vessel, the compliance-enhancing implant device comprising:
a first sealed chamber containing a compressible gas medium; and
one or more second sealed chambers separated from the first sealed chamber by one or more first membrane portions, wherein:
the one or more second sealed chambers contain an at least partially liquid medium;
the first sealed chamber is surrounded on all sides by the one or more second sealed chambers; and
a portion of the compressible gas medium in the first sealed chamber can pass through the one or more first membrane portions into the at least partially liquid medium in the one or more second sealed chambers.

2. The compliance-enhancing implant device of claim 1, wherein the compliance-enhancing implant device is formed as a tubular structure having a central axial flow channel.

3. The compliance-enhancing implant device of claim 2, wherein the one or more second sealed chambers are separated from the axial flow channel by a second membrane portion.

4. The compliance-enhancing implant device of claim 3, wherein the one or more first membrane portions and the second membrane portion are elastic.

5. The compliance-enhancing implant device of claim 1, wherein the one or more second sealed chambers buffer the first sealed chamber from an exterior of the compliance-enhancing implant device.

6. The compliance-enhancing implant device of claim 1, wherein the at least partially liquid medium is saturated with molecules of the compressible gas medium.

7. The compliance-enhancing implant device of claim 1, further comprising an expandable wireform frame.

8. The compliance-enhancing implant device of claim 7, wherein the wireform frame is disposed radially outside of the first sealed chamber and the one or more second sealed chambers with respect to an axis of the compliance-enhancing implant device.

9. The compliance-enhancing implant device of claim 7, wherein the wireform frame is disposed at least partially within the first sealed chamber.

10. The compliance-enhancing implant device of claim 7, wherein the wireform frame is disposed at least partially within the one or more second sealed chambers.

11. The compliance-enhancing implant device of claim 1, wherein:
the compressible gas medium comprises carbon dioxide; and
the at least partially liquid medium comprises saline.

12. The compliance-enhancing implant device of claim 1, wherein the compliance-enhancing implant device has a spheroid structure.

13. The compliance-enhancing implant device of claim 12, wherein:
the first sealed chamber is disposed at an axial center of the spheroid structure; and
the one or more second sealed chambers are disposed radially outside of the first sealed chamber.

14. A compliance-enhancing implant device dimensioned for deployment within a blood vessel, the compliance-enhancing implant device comprising:
a first tubular sealed chamber containing compressible gas; and
a second tubular sealed chamber that covers an inner diameter, outer diameter, and axial end portions of the first tubular sealed chamber, the second tubular sealed chamber being separated from the first tubular sealed chamber by a membrane, wherein the second tubular sealed chamber contains a liquid.

15. The compliance-enhancing implant device of claim 14, further comprising a metal frame disposed radially outside of the second tubular sealed chamber.

16. The compliance-enhancing implant device of claim 14, wherein the liquid contained in the second tubular sealed chamber is saturated with molecules associated with the compressible gas.

17. A compliance-enhancing implant device dimensioned for deployment within a blood vessel, the compliance-enhancing implant device comprising:
a first sealed chamber containing a portion of a compressible gas medium; and
a second sealed chamber separated from the first sealed chamber by a first membrane, wherein:
the second sealed chamber contains an at least partially liquid medium;
the first sealed chamber is surrounded on all sides by the second sealed chamber; and
the at least partially liquid medium within the second sealed chamber is saturated with molecules of the compressible gas medium.

18. The compliance-enhancing implant device of claim 17, wherein the molecules of the compressible gas medium have diffused into the second sealed chamber from the first sealed chamber through the first membrane.

19. The compliance-enhancing implant device of claim 17, wherein:
the first sealed chamber has a spheroid shape; and
the second sealed chamber has a shell form around the spheroid shape of the first sealed chamber.

20. The compliance-enhancing implant device of claim 19, further comprising:
a first stent frame coupled to a first end portion of the shell form of the second sealed chamber; and
a second stent frame coupled to a second end portion of the shell form of the second sealed chamber;
wherein the first and second stent frames are dimensioned to be anchored in a blood vessel lumen and hold the first sealed chamber in alignment with an axis of the blood vessel, such that the second sealed chamber does not contact the blood vessel lumen.
